(19) United States
(12) Patent Application Publication
(10) Pub. No.: US 2002/0081601 A1
(43) Pub. Date: Jun. 27, 2002

(54) Methods and compositions for diagnosis and treatment of cancer based on the transcription factor ets2

(75) Inventors: Dennis K. Watson, Mount Pleasant, SC (US); Tula Christy Papas, Kiawah Island, SC (US)

Correspondence Address: PENNIE AND EDMONDS, 1155 AVENUE OF THE AMERICAS, NEW YORK, NY, 100362711

(73) Assignee: MUSC Foundation For Research Development.

(21) Appl. No.: 09/841,963

(22) Filed: Apr. 25, 2001

Related U.S. Application Data
(63) Continuation of application No. PCT/US99/27805, filed on Nov. 23, 1999.
(60) Provisional application No. 60/109,850, filed on Nov. 25, 1998.

(51) Int. Cl. A61K 48/00; C12Q 1/68; C07H 21/04

(52) U.S. Cl. 435/6; 514/44; 435/325; 536/23.2

(57) ABSTRACT

The present invention relates to methods for treating and preventing cancer by modifying the expression of ets2 gene expression or the activity of the gene product. The invention also relates to sensitizing cancer cells to chemotherapeutic or radiotherapeutic agents. Ets2 gene expression and/or activity of the gene product can be modulated using antisense ets2 nucleic acids and/or modified ets2 proteins. The present invention also provides pharmaceutical compositions which comprise antisense ets2 nucleic acid, and nucleic acid that encode modified ets2 proteins and/or modified ets2 proteins.

This application claims priority to the provisional application No. 60/109,850, filed Nov. 25, 1998, which is incorporated herein by reference in its entirety.

### 1. INTRODUCTION

The present invention relates to methods for treating and preventing cancer based on ets2 that is overexpressed in various cancer tissues. The invention also relates to regulation of gene expression. The invention encompasses ets2 and related nucleic acids, host cell expression systems, mutant ets proteins, ets fusion proteins, antibodies to the gene product, antisense ets2 nucleic acids, and other compounds that modulate gene expression or ets2 activity that can be used for prevention and treatment of cancer disorders, including but not limited to prostate cancer.

### 2. BACKGROUND

### 2.1 Cancer

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis).

Pre-malignant abnormal cell growth is exemplified by hyperplasia, metaplasia, or most particularly, dysplasia (for review of such abnormal growth conditions, see Robbins & Angell, 1976, Basic Pathology, 2d Ed., W. B. Saunders Co., Philadelphia, pp. 68-79.) The neoplastic lesion may evolve clonally and develop an increasing capacity for growth, metastasis, and heterogeneity, especially under conditions in which the neoplastic cells escape the host's immune surveillance (Roitt, I., Brostoff, J and Kale, D., 1993, Immunology, 3rd ed., Mosby, St. Louis, pps. 17.1-17.12).

Clinical data and molecular biologic studies indicate that cancer is a multi-step process that begins with minor preneoplastic changes, which may under certain conditions progress to neoplasia.

Aberrant regulation of the mechanisms that control cell growth and differentiation results in cellular transformation. Molecular analysis has demonstrated that multiple mutations in oncogenes and tumor suppressor genes are required to manifest the malignant phenotype. This multi-step process is well illustrated by colorectal cancers, which typically develop over decades, and appear to require at least seven genetic events for completion (Kinzler et al., 1996, Cell, 87:159-170).

### 2.2 Prostate Cancer

In United States, it is estimated that during the last 15 years of the 20th century, there will be a 37% increase in prostate cancer deaths per year, and a 90% increase in the incidence of disease diagnosed (Carter, H. B. and Coffey, D. S., 1990, The Prostate, 16:39-48). In males, cancer of the prostate now exceeds lung cancer as the most frequently occurring type of cancer, and it is second only to lung cancer in the cause of death due to malignancy (Parker et al., 1997, Ca-A Cancer Journal for Clinicians, 47:5-27). Indeed, for 1997, an estimate of 41,800 deaths and 334,500 new presentations of prostate cancer has been predicted (Parker et al., 1997, Ca-A Cancer Journal for Clinicians, 47:5-17). Although the steady increase in the age adjusted incidence and mortality of this disease is startling, when the increment in the aging population of the U.S. is considered, the dilemma of prostate cancer becomes even more alarming (Carter, H. B. and Coffey, D. S., 1990, The Prostate, 16:39-48 and Bostwick et al., 1992, Cancer Supplement, 70:291-301). Autopsy studies have revealed that as much as 30% of men over the age of 50, and 73% of men over the age of 75 have identifiable prostate carcinomas without having evidenced clinical symptoms (Mostofi et al., 1992, Cancer, 70:235-253).

Although the predominant presentation of prostate malignancy is as a well differentiated, slowly growing tumor in elderly males, at present there is no way to determine whether a tumor will become aggressive and metastasize or remain indolent with little potential for metastasis. Since typical treatment of late stage prostatic malignancy entails chemical or physical castration, and early stage prostatectomy often causes impotency, these procedures often require decisions that greatly influence quality of life. What is needed, aside from the ability to merely detect the presence of malignancy, are clinical markers that distinguish those prostatic carcinomas which are potentially aggressive from those that are unlikely to cause advanced prostate cancer (Mohler et al., 1992, Cancer, 69:511-519). Thus, studying genetic expression at the molecular level in this type of tumor tissue could likely lead to the discovery of genes that might be activated, dysregulated or otherwise essential to the progression of prostatic malignancy and its subsequent metastasis.

### 2.3 ETS Family of Transcription Factors

Alterations in proper control of cellular pathways that regulate cell growth and differentiation can result in cellular transformation leading to cancer. Dysregulation of transcription factor protooncogene expression results in the development of several neoplasias (Clearly, 1991, Cell, 66:619-622). The ETS gene family of sequence-specific transcription factors is an exemplary group (Watson et al., 1988, Proc Natl Acad Sci USA, 85:7862-6 and Watson et al., 1990, Crit Rev Oncog, 1:409-36). All Ets proteins contain a conserved DNA binding domain (Ets domain) of about 85 amino acids that recognizes purine rich sequences containing a -GGAA/T- core (Watson et al., 1990, Crit Rev Oncog, 1:409-36; Bassuk, A. G. and Leiden, J. M., 1997, Advances in Immunology, 64:65-104 and Papas et al., 1997, Leukemia, 11:557-66). Ets proteins have important roles in the transcriptional control of genes important for development, angiogenesis, cell cycle control and cell proliferation. The involvement of Ets genes in cancer was first demonstrated by the presence of Ets sequence in the oncogenic virus, E26 and their importance in human carcinogenesis has been shown by the observation that members of the Ets family are located at the translocation breakpoints of leukemias and solid tumors, forming chimeric proteins with transforming properties (Watson et al., 1990, Crit Rev Oncog, 1:409-36; Bassuk, A. G. and Leiden, J. M., 1997, Advances in Immunology, 64:65-104 and Papas et al., 1997, Leukemia, 11:557-66)8-10).

Ets2 is involved in the regulation of cell division, since it has been shown to be a regulator of cdc-2 (Wen et al., 1995, Experimental Cell Research, 217:8-14) and cyclin D1 (Albanese et al., 1995, Journal of Biological Chemistry, 270:23589-97). Furthermore, increased ets2 expression is observed during liver regeneration following partial hepatectomy (Bhat et al., 1987, Proc Natl Acad Sci USA, 87:3723-7) and after activation of T-cells (Bhat et al., 1990, Proc natl Acad Sci USA, 87:3723-7) and macrophages (Boulukos et al., 1990, Genes Dev., 4:401-9). Ets2 has an important role in signaling response through the CSF-1 (Langer et al., 1992, Mol Cell Biol, 12:5355-62; Sapi et al., 1998, Cancer Research, 58:1027-1033) and Neu/ErbB-2 (Galang et al., 1994, Oncogene, 9:2913-21) cell surface receptors. Inappropriate ets2 expression may lead to cellular transformation: Constitutive ets2 expression transforms NIII3T3 cells, making them tumorigenic (Seth et al., 1989, Proc Natl Acad Sci USA, 86:7833-7). Recently, an elevated level of ets2 has been shown in cervical (Simpson et al., 1997, Oncogene, 14:2149-57) and prostate cancer (Liu et al., 1997, The Prostate, 30:145-153). However, the involvement of ets2 genes in various human cancers remains unexplored.

Although the molecular etiology of prostate cancer is not defined, several genetic alterations have been detected. In addition, gene amplification plays a role in some prostate cancers (Cheng et al., 1996, Proc. Natl. Acad. Sci., USA, 93:3636-3641). However, these multiple parameters remain poorly correlated with the molecular events associated with a multi-step progression of the malignancy. Thus, there is a great need for additional data on the expression of specific proteins associated with prostate cancer which would facilitate a better understanding of the molecular biology of prostate cancer, and provide the information to develop novel therapeutics.

### 3. SUMMARY OF THE INVENTION

The present invention relates to the discovery that inhibition of ets2 functions in cancer cells, prostate cancer cells in particular, leads to a reduction of the transformed properties of the cancer cells.

Ets2 transcripts were detected in two high-grade human prostate cancer cell lines. Inhibition of ets2 expression by antisense RNA or expression of a dominant negative ets2 mutant reduced the transformed phenotype of these two prostate cancer cell lines. As such, the ets2 gene product are involved in the mechanisms underlying the onset and development of prostate cancer as well as the infiltration and metastatic spread of cancer.

In particular, the invention provides methods for using antisense ets2 nucleic acids and/or mutant ets2 gene products to modulate the expression of ets2 gene and/or the activity of ets2 gene product in cancer cells. The ets2 gene is a transcription factor the expression of which is abnormal in various cancer cell lines and tissues. Thus, the present invention also provides methods for the prevention and/or treatment of cancer, and for the control of metastatic spread of cancer, that are based on modulation of the expression and activity of ets2 gene or gene product.

Moreover, the present invention provides compositions which encompass nucleic acid molecules that encode ets2 mutant proteins and ets2-repressor fusion proteins, degenerate variants thereof, and naturally occurring variants thereof, as well as antisense ets2 nucleic acid molecules. The compositions of the present invention additionally include cloning vectors, including expression vectors, containing the nucleic acid molecules of the invention, and hosts which contain such nucleic acid molecules. The compositions of the present invention also encompass ets2 mutant gene products, variants and fragments thereof, ets2-repressor fusion proteins, and antibodies directed against ets2.

Further, methods and compositions are presented for the treatment or prevention of cancer, especially prostate cancer. Such methods and compositions are capable of modulating the level of ets2 gene expression and/or the level of ets2 gene product activity in a patient's cell. Such compositions can also be used to palliate the symptoms of the disease, and control the metastatic potential of the cancer.

Still further, the present invention provides methods for sensitizing cancer cells to methods of cancer treatment or prevention by modulating the expression of ets2 gene and/or the activity of ets2 gene product in cancer cells. The present invention also provides methods for treatment or prevention of cancer comprising administering the claimed compositions prior to, simultaneous with, or after, using other cancer treatment or prevention methods.

The inventors tested the sensitivity of cancer cells to chemotherapy wherein the ets2 protein activity of the cells had been modulated. When exposed to cisplatin, prostate cancer cells that express antisense ets2 RNA have a lower percentage of survival than prostate cancer cells that do not express the antisense ets2 molecule. Accordingly, in another embodiment the methods of the invention can also be used to sensitize cancer cells to chemotherapeutic agents, such as cisplatin.

### 4. BRIEF DESCRIPTION OF THE FIGURES

FIG. 1. Ets2 expression in human prostate cancer cell lines. Total RNA (5 μg/lane) was electrophoresed on 1.2% agarose containing formaldehyde, transferred to a nylon membrane and hybridized with ³²P-labeled ets2 probe. (A) Ethidium bromide stained RNA; (B) Northern Blot analysis; Positions of 28S and 18S rRNA and calculated sizes of ets2 transcripts are indicated.

FIG. 2. Identification of ets2 transfected cell lines. Total RNA (5 μg/lane) prepared from PC3, and DU145 parental and selected transfected cell lines was electrophoresed on 1.2% agarose containing formaldehyde, transferred to a nylon membrane and hybridized with ³²-P labeled ets2 probe. Reduction of endogenous ets2 expression in stable antisense transfectants of PC3 and DU145 is observed.

FIG. 3. Reduction of anchorage-independent growth in antisense ets2 transfectants. Soft agar colonies formed after three weeks from (A) parental DU145 and two antisense transfectant clones (DUα2O, DUα21) and (B) parental PC3 and two PC3 antisense transfectants (PCα2, PCα4). The histograms show the number of the soft agar colonies, by size. Colonies were photographed and representative fields are shown (insert).

FIG. 4. Identification of cell lines expressing DN-ets2. (A) Total RNA (5 μg/lane) prepared from parental and selected transfected cell lines was electrophoresed on 1.2% agarose containing formaldehyde, transferred to a nylon membrane and hybridized with ³²-P labeled ets2 probe. Positions of endogenous ets2 mRNA (ets2, 4.7 and 3.2 kb) and exogenous, mutant ets2 (Ets2/DN) mRNA (1.0 kb) are indicated. Clones expressing the smaller, exogenous ets2 mRNA were selected and further analyzed. (B) Protein was prepared from metabolically labelled cells and incubated with pan ets (a) or C20 (b) antibody. Washed immunoprecipitates were resolved on 10% SDS-PAGE and detected by fluorography. The relative mobility of a 16 kD protein size marker is indicated by the arrow.

FIG. 5. Mutant ets2 (DN-ets2) inhibits anchorage-independent growth. Soft agar colony assays for parental DU145 and DN-ets2 transfectants (DU DN14, DU DN21) were quantitated after three weeks.

FIG. 6. Nucleotide sequence of ets2 cDNA.

FIG. 7. Amino acid sequence of ets2 protein.

FIG. 8. Alignment and Features of ets domains. The ets domain sequences were aligned with the clustal program (Higgins and Sharp, 1998, Gene, 73:237-244). Amino acids that are matched across all sequences are indicated by “*”, and conservative substitutions by “●”. Amino acids that match Etsl are shaded. The references for the sequences are: human Etsl (ETS1 HUMAN, Watson et al., 1988, Proc. Natl. Acad. Sci. USA, 85:7862-7866; Reddy and Rao, 1988, Oncogene Res., 3:239-246), mouse Etsl (EST1 MOUSE, Chen, 1990, Oncogene Res., 5:277-285; Gunther et al., 1990, Genes Dev., 4:667-679), chicken Ets1 p68 (ESTB CHICK, LePrince et al., 1988, Oncogene, 7:9-17; Watson et al., 1988, Virology, 164:99-105) chicken Ets1 p54 (ETSA CHICK, Chen, 1988, Oncogene Res., 2:371-384; Duterqe-Coquillaud, 1988, Oncogene Res., 2:335-344; Watson et al., 1988b, Virology, 164:99-105; the ets domain is identical to ETSB CHICK and not shown), X. laevis Ets1 (ETSA XENLA, Stiegler et al, 1990, Nucleic Acids Res., 18:5298), vEts from E26 virus (vETS E26, Nunn et al., 1983, Nature, 306:391-395; Golay et al., 1988, Cell, 55:1147-1158), X. laevis Ets2 (ETS2 XENLA, Burdett et al., 1992, Nucleic Acids Res., 20:371; Wolff et al., 1991, Cell Growth Differ., 2:447-456), human Ets2 (ETS2 HUMAN, Watson et al., 1988a, Proc. Natl. Acad. Sci. USA, 85:7862-7866), mouse Ets2 (ETS2 MOUSE, Watson et al., 1988a, Proc. Natl. Acad. Sci. USA, 85:7862-7866), chicken Ets2 (ETS2 CHICK, Boulukos et al., 1988, Mol. Cell Biol., 9:5718-5721), sea urchin Ets2 (ETS2 SEAUR, Chen et al., 1988, Dev. Biol., 125:432-440), D. melanogaster Ets2 (ETS2 DROME, Pribyl et al., 1988, Dev. Biol., 127:45-53), Fli (FLI MOUSE, Ben-David et al., 1991, Genes Dev., 5:908-918), Erg1+2 (ERG HUMAN, Reddy et al., 1987, Proc. Natl. Acad. Sci. USA, 84:6131-6135), Drosophila Ets3 and Ets6 (ETS3 DROME, Chen et al., 1992a, Dev. Biol., 151:176-191), GABP RAT, LaMarco et al., 1991, Science, 253:789-792), D-Elg (DELG DROME, Pribyl et al., 1991, Oncogene, 6:1175-1183), PEA3 (PEA3 MOUSE, Xin et al., 1992, Genes. Dev., 6:481-496), Elk1+2 (ELK HUMAN, Rao et al., 1989, Science, 244:66-70), SAP-1a and b (SAP1 HUMAN, Dalton and Treisman, 1992, Cell, 68:597-612), ElkX (ELKX MOUSE), Elf1 (ELF1 HUMAN, Thompson et al., 1992, Trends Genet., 8:232-236), E74A and B (E74A DROME, Burtis et al., 1990, Cell, 61:85-99), D. melanogaster Ets4 (ETS4 DROME, Chen et al., 1992a, Deve. Biol., 151:176-191), PU1 (PU1 MOUSE, PU1 HUMAN, Moreau-Gachelin et al., 1989, Oncogene, 4:1449-1456; Moreau-Gachelin et al., 1990, Leukemia, 4:20-23; Ray et al., 1990, Oncogene, 5:663-668; Klemsz et al., 1990, Cell, 61:113-124; Paul et al., 1991, J. Virol., 65:464-467). The features indicated are the tryptophan repeat (Anton and Frampton, 1988, Nature, 336:719), basic region, and predicted structures including an α-helix (Wang et al., 1992, J. Exp. Med., 175:1391-1399), a helix-loop-helix (Rao and Reddy, 1992, Oncogene, 7:65-70) and a βturn/α-helix (Seth et al., 1990, Oncogene, 5:1761-1767).

FIG. 9A-B. Ets expression is elevated in drug-resistant cell lines. Expression of ets2 and etsl transcripts in 289, 289T and 289D cell lines. RNA from parental melanoma cell line 289 (left lane), from tamoxifen-resistant cell line 289T (middle lane), and from cisplatinresistant cell line 289D (right lane) were analyzed by northern hybridization with a radiolabeled ets2 probe. Ethidium bromide-stained agarose gels before blotting are shown below each autoradiogram. Names of cell lines are indicated above each lane.

FIG. 10. Ets2 confers resistance to cisplatin [cis-diamminedichloroplatinum(II)]. Increased sensitivity of cell lines expressing antisense ets2 RNA to cisplatin [cis-diamminedichloroplatinum (II)] is depicted as % survival of (i) DU145 cancer cells (lines with solid diamonds), (ii) DU20 cells which are derived from DU145 cells by transformation with gene construct expressing antisense ets2 RNA (lines with triangles), and (iii) DU21 cells which are derived from DU145 cells by transformation with gene construct expressing antisense ets2 RNA (lines with solid circles), as a function of the concentration of cisplatin [cis-diamminedichloroplatinum (II)].

### 5. DETAILED DESCRIPTION OF THE INVENTION

The present invention relates to methods for reducing the tumorigenicity of preneoplastic and neoplastic cells, methods for treating and/or preventing cancer, and methods for palliating the symptoms of cancer. The invention also encompasses methods for inhibiting the transcriptional activity of an ets protein, and methods for inhibiting the expression of genes that are under the transcriptional control of ets proteins. The invention also provides methods of treatment and prevention of cancer in a subject, particularly in a human. In particular, the invention provides methods for inhibiting ets2 gene function in prostate cancer cells. Compositions useful in these methods are also encompassed by the invention.

Prostate cancer has been difficult to manage clinically because it yields few clues that portend aggressive behavior. Cancer is the result of the accumulation of multiple genetic changes, resulting in either the loss of expression of tumor suppressor genes or the overexpression of oncogenes. While a number of important molecular genetic alterations have been reported in recent years (Isaacs, J. T., 1997, American Journal of Pathology, 150:1511-21), it has been difficult to correlate changes in these genes to the onset of an aggressive clinical course. In fact, a large number of genetic defects accumulate at a greater rate than can be explained by ordinary mutation rates. Given this accelerated rate of genetic alterations, the inventors recognized that the stochastic nature of prostate tumor progression could be due in part to the dysregulation of transcription factor expression. Alterations in a transcription factor are likely to affect many other genes, some of which ultimately lead to the outgrowth of cells whose normal controls on growth and invasiveness has been compromised.

Accordingly, the inventors set out to assess the contribution of one member of the ETS gene family, ets2, to the phenotype of cancer cells, and prostate cancer cells in particular. Ets2 transcripts were detected in high-grade human prostate cancer cell lines. Transfection of these prostate cancer cell lines provides a model system in which one can manipulate ets2 expression to determine its contribution to the transformed phenotype. Two approaches to blocking ets2 function in prostate cancer cells have been developed. Both approaches resulted in cell lines that no longer have functional ets2, and have a much reduced transformed phenotype, i.e., the ability to grow in an anchorage independent manner when compared to the parental cell lines.

In one study, the inventors generated clonal cell lines that express an antisense ets2 construct. The antisense ets2 construct hybridizes to messenger RNA (mRNA) encoding ets2 gene product and blocks translation of the ets2 MnRNA. When an excess of antisense ets2 nucleic acid molecules is present, it can hybridize with, and thereby inactivate most of the ets2 RNA in a cell. Thus, in one embodiment, the tumorigenicity of prostate cancer cells can be reduced by the expression of an ets2 antisense nucleic acid molecule.

In another study, the inventors inhibited ets2 function as a transcription factor by creating prostate cell clones that expresses a dominant negative mutant of ets2. The mutant ets2 gene product which is inactive, competes with endogenous wild type ets2 protein for ETS binding sites, and effectively blocks the binding of functional wild type ets2 protein. As used herein, the term “dominant negative”, when used in reference to ets2 mutant gene products of the present invention, refers to species of ets2 mutant gene products which have a negative effect on the transcription of a ets2 target gene, even in the presence of wild type ets2 gene products. Accordingly, in another embodiment of the invention, the tumorigenicity of prostate cancer cells can be reduced by the expression of a dominant negative mutant of ets2 gene product.

The inventors have also demonstrated that de novo expression of ets2 in a weakly tumorigenic human prostate cell line increases its ability to grow in soft agar. The observation supports the rationale of the invention that ets2 contributes to and is necessary for the maintenance of the transformed phenotype of prostate cancer cells. Thus, the inhibition of ets2 expression or activity in prostate cancer cells can lead to a reduction of tumor phenotype of the cancer cells.

It has also been demonstrated that ets2 expression is elevated in a range of cancer cells, such as but not limited to cervical cancer cells. In view of the results obtained with prostate cancer cells, the inventor recognizes that dysfunction of the ets2 gene control in these cancer cells generally contributes to their tumorigenicity and metastatic potential, and that inhibition of ets2 function can reduce in these cancer cells tumorigenicity and the likelihood of metastasis. Accordingly, the present invention also encompasses methods for reducing tumorigenicity of cancer cells in which the activity of ets2 is elevated, said methods comprising inhibiting the expression or activity of ets2 in said cancer cells.

The dominant negative mutant ets2 gene products of the invention can be used to reduce tumorigenicity as well as the metastatic potential of any said cancer cells.

The dominant negative mutant ets2 gene products of the invention can also be used to reduce the transformed phenotype of cancer cells with the proviso that the transformed phenotype of the cancer cells is not stimulated by colony-stimulating factor-1 (CSF-1).

Furthermore, ets2 protein is a transcription factor that regulates expression of a number of genes, herein referred to as ets2 target genes. Some of these genes are expressed or upregulated in aggressive prostate cancer cells. The expression of some of these ets2 target genes contributes to the metastatic potential of cancer cells, and the cancerous phenotype of preneoplastic and neoplastic cells. Thus, in yet another embodiment, the invention provides methods for regulating the transcription of these ets2 target genes in preneoplastic and neoplastic cells. The genetically engineered preneoplastic and neoplastic cell lines of the invention can also facilitate identification of ets2 target genes that are associated with cancer development and progression.

For more efficient blocking of the transcription activity of endogenous ets2 protein, the invention have further provided novel fusion proteins comprising a dominant negative mutant of ets2 and a transcriptional repressor. These novel fusion proteins can be expressed in the preneoplastic and neoplastic cells to block the interaction of the, endogenous ets2 protein and its binding sites in the 5′ end of various ets2 target genes.

Due to the similarity in structure of ETS binding sites, it is also contemplated that dominant negative ets2 mutants of the invention can also inhibit the transcriptional activity of other ETS family members. Thus, in addition to inhibiting the transcriptional targets of ets2, it is expected that the dominant negative ets2 mutants would also block the transcriptional activity of other closely related ETS family members, such as etsl. For example, etsl is also expressed in prostate cancer cell lines.

As it has been demonstrated that dominant negative mutants of ets2 are capable of inhibiting the transcriptional activity of ets2, the inventors provided that this approach can be broadly applied to the inhibition of transcription activity of other ETS family members. In this embodiment of the invention, fusion proteins comprising a dominant negative mutant of a ETS family member and a transcriptional repressor is provided. These novel fusion proteins can be expressed in the preneoplastic and neoplastic cells to block the interaction of the endogenous ETS protein and its binding sites.

Accordingly, the interference of the transcriptional activity of an ets protein may provide a novel therapeutic approach for cancers and other disease conditions that are associated with excess transcriptional activity of an ets protein, or overexpression of an ets protein.

The present invention also encompasses (a) DNA vectors that contain any of the foregoing antisense ets2 gene, and modified ets2 gene sequences encoding mutant and fusion ets proteins; (b) DNA expression vectors that contain any of the foregoing antisense ets2 gene, and modified ets2 gene sequences encoding mutant and fusion ets proteins operatively associated with a regulatory element that directs the transcription and/or expression of the foregoing antisense ets2 gene, and modified ets2 gene sequences encoding mutant and fusion ets proteins; and (c) genetically engineered host cells that contain any of the foregoing DNA vectors or DNA expression vectors. As used herein, regulatory elements include, but are not limited to inducible and non-inducible promoters, enhancers, operators and other elements known to those skilled in the art that drive and regulate expression.

In various embodiments as described above, inhibition of ets2 expression or activity in neoplastic cells can lead to a less aggressive phenotype, which in turn could result in tumor regression, reduced metastasis, and/or reduced malignancy. Inhibition of ets2 expression or activity in preneoplastic cells can lead to the non-development of a neoplastic lesion. Thus, the methods of the invention can also be used for treating and/or preventing cancer, as well as for palliating the symptoms of cancer.

As used herein, cancer cells include neoplastic cells, and preneoplastic cells. Preneoplastic cells include cells that are infected with a cancer-causing infectious agent, such as a virus, but which are not yet neoplastic; or cells that have been exposed to a mutagen or cancer-causing agent, such as, for example DNA-damaging agents, radiation, etc. Other preneoplastic cells that are encompassed are cells which are in transition from a normal to a neoplastic form as characterized by morphology, cytogenetics, physiological or biochemical functions. Preferably, the cancer cells and preneoplastic cells are of mammalian origin. Mammals contemplated by this aspect of the invention include humans, companion animals (e.g., dogs and cats), livestock animals (e.g., sheep, cattle, goats, pigs and horses), laboratory animals (e.g., mice, rats and rabbits), and captive or free wild animals.

In various embodiments, any cancer cells, preferably human cancer cells, can be treated by the present methods for reducing its tumorigenicity, metastatic potential, or transformed phenotype. Cancers which can be treated or prevented with compositions prepared by methods of the invention include, but are not limited to, tumors such as sarcomas and carcinomas. Examples of cancers that are amenable to the methods of the invention are listed in Section 5.5. Accordingly, any tissues or cells in a preneoplastic lesion, a cancer, including cancer that has metastasized to multiple remote sites, can be treated by the methods of the invention. For example, cells found in abnormally growing tissues, solid tumor tissues, metastatic lesions, as well as circulating leukemic cells, can be used.

In another embodiment, cell cultures or cell lines derived from a preneoplastic lesion, cancer tissues or cancer cells can be used to test and fine-tune the efficacies of the various compositions of the invention, including but not limited to antisense ets2 RNA and modified ets2 proteins. Preferably, cancer cells are used that are excised from the patient to which ultimately the antisense ets2 RNA or modified ets2 are to be administered, although this need not be the case (e.g., the cancer cells can be from one or more different individuals).

Cancer and preneoplastic cells can be identified by any method known in the art. For example, cancer cells can be identified by morphology, enzyme assays, proliferation assays, cytogenetic characterization, DNA mapping, DNA sequencing, the presence of cancer-causing virus, or a history of exposure to mutagen or cancer-causing agent, imaging, etc. As for another example, cancer cells can be obtained by surgery, endoscopy, or other biopsy techniques. If some distinctive characteristics of the cancer cells are known, they can also be obtained or purified by any biochemical or immunological methods known in-the art, such as but not limited to affinity chromatography, and fluorescence activated cell sorting (e.g., with fluorescently tagged antibody against an antigen expressed by the cancer cells).

### 5.1 The ETS Family

The Ets family encompasses a large number of genes that encode transcription factors and share a common DNA-binding domain (Ets domain), which recognizes a GGAA purine-rich core sequence found in the promoters or enhancers of a variety of genes. (Graves & Petersen, 1998, Advances in Cancer Research, Vol. 75: 1-55, Bhat et al., 1996, Int. J. Oncol, 8: 841-846, Dittmer and Nordheim, 1998, Biochimica et Biophysica Acta, 1377: 1-11, and Yaniv and Ghysdael eds, 1997, Oncogenes as Transcriptional Regulators, Birkhauser Verlag: Basel, at page 29-88). In addition, ets transcription factors also play important roles in the development and function of the mammalian immune system (Bassuk and Leiden, 1997, Advances in Immunology, 64: 65-104).

Members of the ets family of proteins can be subdivided into three classes by the position of the consensus ets domain in the protein (see Janknecht & Nordheim, Biochimica et Biophysica Acta 1155(1993)346-356): a majority with a carboxy terminal ets domain including but not limited to ets1, ets2, erg, PEA3, GABPα, ER71, ER81, PU.1, Fli-1, E74, -Elg, pointed; a class with a central ets domain, such as but not limited to elf-1 and Pok/Yan, and a class with amino terminal ets domain, such as but not limited to elk-1, SAP1, and SAP-2.

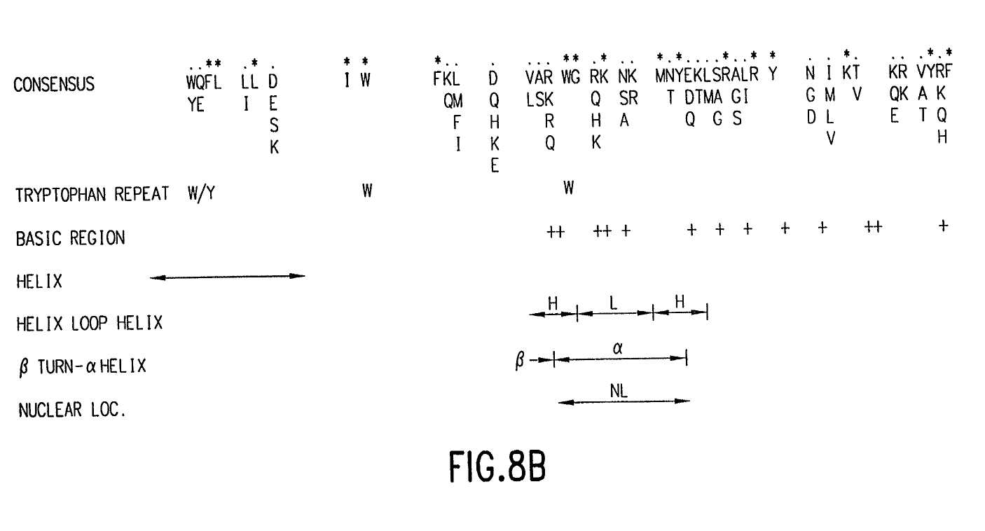

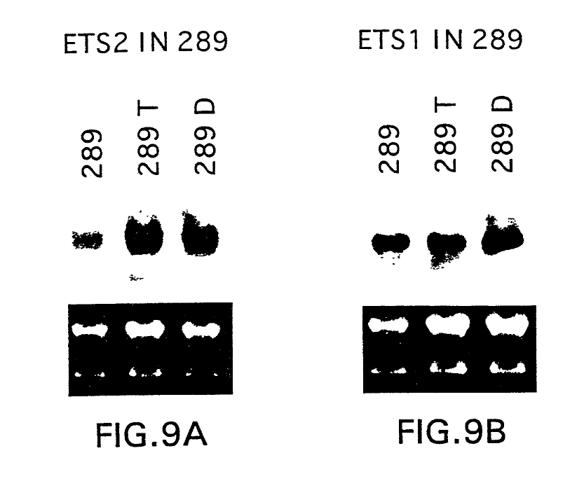

As expected, the ets domain in ets2 shares significant sequence homology with other members of the ets family of transcription factors. An alignment of the ets domains of exemplary members of the ets family is provided in FIG. 8. The ets domain is required for specific DNA binding: Est1, Ets2 (Wasylyk et al., 1992, Genes Dev. 6:965-974); GABPα, (Thompson et al., 1991, Science 253:762-768); PEA3 (Xin et al., 1992, Genes Dev. 6:481-496); SAP1 (Dalton and Treisman, 1992, Cell 68:597-612); PU1 (Wasylyk et al., 1992, supra). It has a unique structural motif for specific DNA binding, since the pattern of contacts with DNA is distinct from other transcription factors. It lacks the classical features of other transcription factor families (e.g. homeo-domain, helix-turn-helix, zinc fingers, basic-leucine repeat, basic-helix-turn-helix, rel domain), although a number of potential structural motifs have been noted. The tryptophan repeat resembles that of the myb DNA-binding domain and it is thought to be important for DNA binding. There is also a basic region, a feature of many DNA binding domains. Structural analysis of members of the family predicts an a-helical region surrounding the first tryptophan and a helix-loop-helix or a β-turn/α-helix surrounding the third tryptophan. A number of additional sequence similarities in the ets domain have also been predicted by Seth et al. (1990, Oncogene 5:1761-1767), including a nuclear localization signal, a cell-division motif, and an ATP-binding domain.

### 5.1.1 Ets2 Proteins And Genes

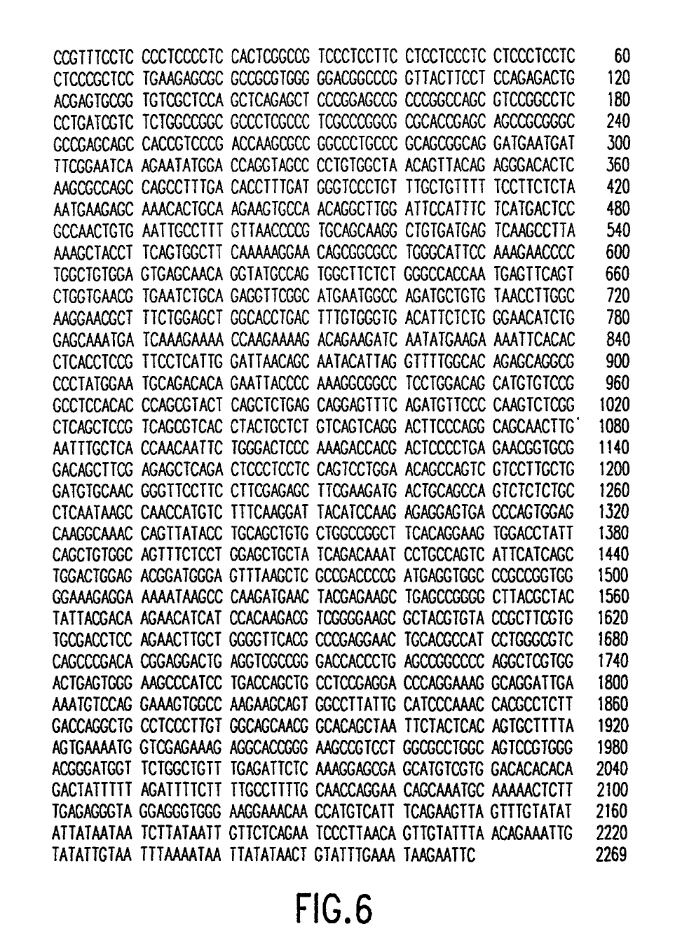

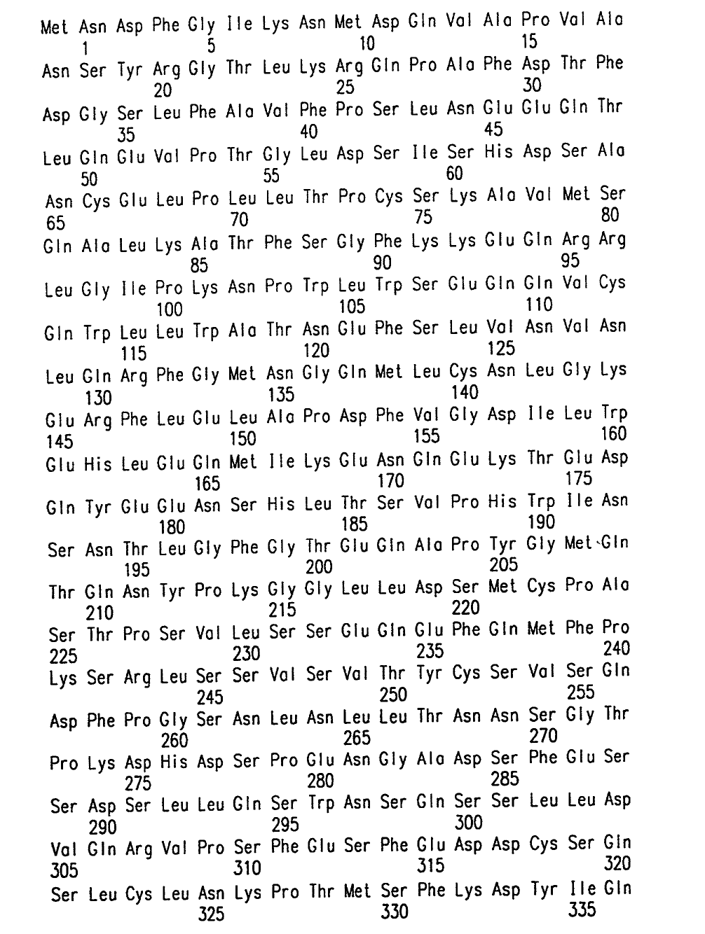

The published full length human ets2 complementary DNA (cDNA) as depicted in FIG. 6 consists of 2269 nucleotides (Watson et al., 1988, Proc. Nati. Acad. Sci. USA, 85:7862-7866). The coding region of the human ets2 cDNA (nucleotide 292-1701) encodes a polypeptide of 469 amino acids. The human ets2 gene has been cloned and is localized to the long arm of chromosome 21 (21q22.3) (Watson et al., 1985, Proc. Natl. Acad. Sci. USA, 82:7294-7298). The coding sequence for ets2 have also been cloned and sequenced in a variety of organisms including Xenopus laevis, chicken, mouse, sea urchin, and Drosophila melanogaster (Wasylyk et al.,1993, Eur. J. Biochem. 211:7-18), and were found to be highly conserved. The nucleic acid sequences of the human ets2 cDNA and human ets2 gene are deposited with Genbank and given respectively the Accession Numbers J04102 and M11922.

The ets2 protein (or ets2 gene product) as depicted in FIG. 6 comprises 469 amino acids and has a molecular weight of 56,000 daltons (Watson et al., 1988, Proc. Natl. Acad. Sci. USA, 85:7862-7866). The published amino acid sequence of human ets2 protein has been deposited with GenBank and given the Accession Number 182273. The amino acid sequence of the full length ets2 gene product comprises two activation domains, and a DNA binding domain, which is referred to herein as the ets2 domain.

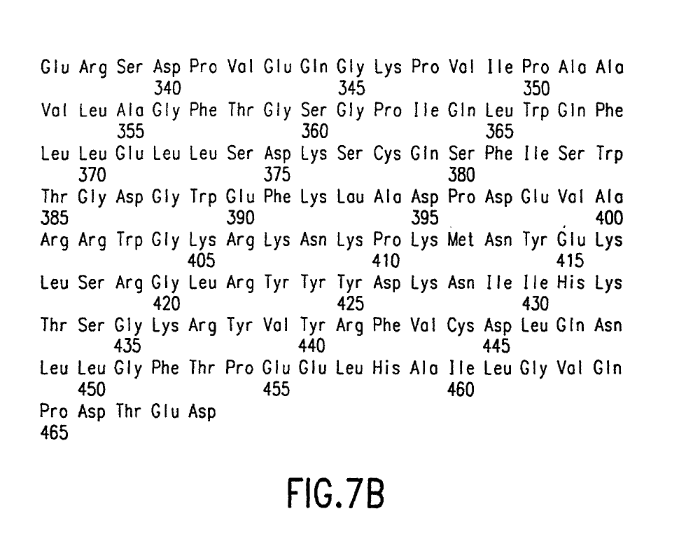

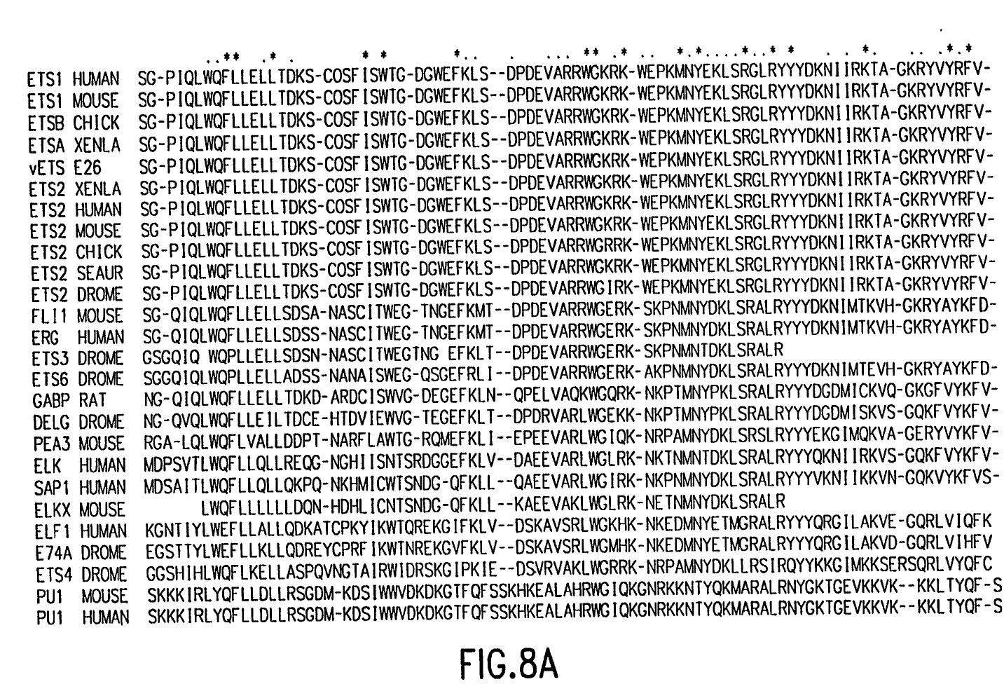

As used herein, “ets2 gene” refers to (a) a nucleic acid molecule comprising the DNA sequence shown in FIG. 6 or designated Genebank Accession No. J04102; (b) any nucleic acid molecule having a DNA sequence that encodes the amino acid sequence shown in FIG. 7 or designated GenBank Accession No. 182273; (c) a nucleic acid molecule that hybridizes to another nucleic acid consisting of the complement of the DNA sequences that encode the amino acid sequence shown in FIG. 7 or designated GenBank Accession No. 182273, under highly stringent conditions, e.g., hybridization to filter-bound

DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at page 2.10.3).

The term “ets2 gene” also includes naturally occurring variants of ets2, and degenerate variants of DNA sequences of (a) through (c) as described above. A ets2 gene sequence preferably exhibits at least about 80% overall similarity at the nucleotide level to the nucleic acid sequence depicted in FIG. 6, more preferably exhibits at least about 85-90% overall similarity to the nucleic acid sequence in FIG. 6 and most preferably exhibits at least about 95% overall similarity to the nucleic acid sequence in FIG. 6. The degree of similaritiy can be determined by analyzing sequence data using a computer algorithm, such as those used by the BLAST computer program. The ets2 gene may be a segment of the cDNA molecule, or a genomic DNA molecule that comprises one or more intervening sequences or introns, as well as regulating regions located beyond the 5′ and 3′ ends of the coding region or within an intron.

### 5.1.2 Ets2 Antisense Molecules

In one embodiment, the invention provides antisense ets2 nucleic acid molecules, preferably RNA molecules, that are essentially single stranded nucleic acid molecules, and comprises a nucleotide sequence complementary to (a) the nucleotide sequence of the sense strand of the polynucleotide depicted in FIG. 6, or designated Genebank Accession No. J04102; or (b) a nucleotide seqeunce that encodes the amino acid sequence shown in FIG. 7 or designated GenBank Accession No. 182273.

The antisense ets2 nucleic acid molecule of the invention is capable of hybridizing in vivo and in vitro to a portion of an ets2 messenger RNA (mRNA) by virtue of some sequence complementarity. Such hybridization conditions may be highly stringent as exemplified above, or moderately stringent, e.g., washing in 0.2×SSC/0.1% SDS at 42° C. (Ausubel F. M. et al., eds., 1989, Current Protocols in Molecular Biology, Vol. I, Green Publishing Associates, Inc., and John Wiley & sons, Inc., New York, at page 2.10.3). In instances where the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C (for 20-base oligos), and 60° C. (for 23-base oligos).

Antisense nucleic acid molecules may be synthesized chemically or enzymatically, and delivered to cancer cells by injection. Alternatively, antisense ets2 RNA molecules can be synthesized in a cell by inserting the ets2 gene or a fragment thereof in a manner such that the antisense RNA molecules are made, preferably in a controllable fashion. Furthermore, double-stranded RNA which has been shown to effectively block gene expression can also be used. Genetic interference by double-stranded RNA (RNA interference or RNA-i) has been successfully used to determine both the role of a specific gene and cells that express the specific gene (Misquitta and Paterson, 1999, Proc. Natl. Acad. Sci., 96: 1451-1456; Fire et al., 1998, Nature, 391: 806-811).

These nucleic acid molecules may be used to interfere with ets2 gene regulation, so as to modulate, for example, the phenotype and metastatic potential of preneoplastic and neoplastic cells. Further, such sequences may be used as part of ribozyme and/or triple helix sequences, also useful for ets2 gene regulation. Details on the use of antisense ets2 molecules, ribozymes, and triple helix sequences are provided in Section 5.7.

### 5.1.3 Ets2 Dominant Negative Mutants

In another embodiment, the invention provides dominant negative mutants of ets2 protein which include fragments of the ets2 protein, and truncated ets2 protein. In a preferred embodiment, the ets2 fragments correspond to the DNA binding domain of ets2 including the NL signal. Truncated ets2 in which one or more activation domain(s) is deleted are also preferred. Less preferred are dominant negative mutants of ets2 in which one or more amino acid(s) are substituted or deleted within the one or both activation domain(s).

The DNA binding domain of ets2, or the ets2 domain comprises about 85 amino acids, i.e., from about amino acid position 361 to about amino acid position 446, that recognizes a block of purine rich sequences that is about 10 bp in length and has in the middle the invariant core sequence C/A GGA A/T (Watson et al., 1990, Crit. Rev. Oncog. 1:409-36; Bassuk, A. G. and Leiden, J. M., 1997, Advances in Immunology, 64:65-104 and Papas et al., 1997, Leukemia, 11:557-66). DNA motifs to which ets2 binds are herein referred to as ets2 target sites, and include but are not limited to GCAGGAAGTG, GCAGGAAGCA, CCAGGAAATG, CCAGGAAGTG.

The ets2 domain, like the other ets domains can be subdivided into a region containing three highly conserved tryptophans, separated by about 17-21 amino acids and a basic region. The ets2 domain further comprises a nuclear localization (NL) signal sequence. The key features that constitute the consensus ets domain are retained in the ets2 domain, e.g., the three highly conserved tryptophan residues which are present at amino acid positions 366, 384 and 403 in ets2.

The dominant negative mutants of ets2 are capable of binding to ets2 target sites, but incapable of activating transcription of the ets2 target gene at normal level. Preferably, the dominant negative mutants of ets2 suppress transcription of ets2 target genes.

Accordingly, the dominant negative mutants of ets2 of the invention have substitutions, deletions, and/or insertions, of one or more amino acid residues in the wild type ets2 protein. Preferably, the substitutions, deletions, and/or insertions are made in regions that are related to the transcription activation functions of ets2.

### 5.1.4 Repressor Elements

The invention further provides ets2 fusion proteins comprising a dominant negative mutant of ets2 and a repressor element derived from a transcription repressor. Also provided are nucleic acid molecules encoding ets2-repressor fusion proteins.

Repressor elements are found in proteins that exhibit inhibitory activity towards initiation of transcription of a gene. A significant number of proteins that are capable of functioning as transcriptional repressors have been identified, and many of them are known to play key roles in a variety of cellular and developmental processes. A number of repressors have been shown to consist of a modular structure, i.e., they contain separable DNA binding and repression elements. This was shown first with the Krüppel protein, which contains DNA binding zinc fingers and a distinct region that is capable of blocking transcription in transfected mammalian cells when fused to a heterologous DNA binding domain (Licht et al., 1990, Nature 346:76-79). The KRAB domain (Krüppel-associated box) which consists of about 75 amino acids, is a repression element that is found in many cys2-his2 zinc finger proteins. The KRAB doamin can be further subdivided into A and B boxes by common intron-exon boundaries of related members of the family. The minimal KRAB domain with repressor activity is a block of about 45 amino acids localized to the KRAB-A box (Margolin et al., 1994, Proc. Natl. Acad. Sci. 91:4509-4513; Friedman et al., 1996, Genes & Development, 10:2067-78). This block of about 45 amino acids can be used as a repressor element of the invention.

Many other transferable repression elements known in the art can also be used in the present invention. These include but are not limited to, for example, the homeodomain protein α2, which functions with other proteins to control cell type in yeast (Keleher et al. 1988); the homeodomain proteins Even-skipped (Eve; Han & Manley, 1993 Genes Dev. 7, 491-503) and Engrailed (En; Jaynes & O'Farrell 1991, EMBO J. 10:1427-33; Han & Manley,1993, EMBO J. 12:2723-2733), which are involved in pattern formation during early Drosophila embryogenesis; and in mammals, the zinc finger-containing v-erbA oncoprotein or thyroid hormone receptor (Baniahmad et al., 1992, EMBO J. 11:1015-1023), and the WT1 Wilms tumor gene product (Madden et al., 1991, Science 253:1550-1553; Gashler et al., 1992, Proc. Natl. Acad. Sci. 89:10984-8; and Wang et al.,1993, J. Biol. Chem. 268:9172-5). In YY1, the repressor element is localized to four carboxyl-terminal zinc fingers (Galvin and Shi, Mol. Cell Biol. 1997,17:3723). In human thyroid hormone receptor beta, repression activity is mediated by the amino-terminal region and the ligand binding domain (CoR box).

Another family of transferable repressor elements that can be used to make the ets2-repressor fusion protein is found in the protooncogene Gfi-1 (Grimes et al., 1996, Mol. Cell Biol. 16:6263-6272). This element consisting of about 20 amino acids, is coincident with a nuclear localization signal in the amino terminal of the protein. This repressor element, also known as the SNAG domain, is evolutionarily conserved and is shared by Gfi-1B, the orphan Hox gene Gsh-1, the insulinoma-associated protein (IA-1), and the vertebrate (but not Drosophila) members of the Snail/Slug protein family.

Repressor elements useful in the present invention can be identified and localized by fusing portions of a transcription repressor to a defined DNA binding domain, such as but not limited to GAL4, and determining the transcriptional activity of the fusion protein (Madden et al., 1991, Science 253:1550-1553).

The primary amino acid sequences of the different classes of repressor elements are different; and they may repress initiation of transcription by different mechanisms. Without being bound by any particular theory, the repressor elements useful in the invention may exert its repressive activity employing one of several distinct mechanisms (reviewed in Johnson, 1995, Cell 81:655-658). The simplest involves competition for DNA binding sites, whereby the repressor interferes with binding of either an activator or basal transcription factor, by virtue of adjacent or overlapping binding sites. A second mechanism, known in the art as quenching, involves simultaneous DNA binding both the activator and repressor, coupled with a protein-protein interaction that prevents the activator from functioning, for example by masking the activation domain. Thirdly, a direct repressor functions by binding DNA and then interfering, via protein-protein interactions, with the formation or activity of the basal transcription complex. This form of repression would appear to be analogous to those thought to be employed by transcriptional activators, except leading to repression rather than activation of transcription. The thyroid hormone receptor, the Drosophila Krüppel protein, and Eve appear to function as direct repressors. A fourth mechanism involves histone proteins wherein the repressor recruits histone acetylase to regions of the chromatin where it irreversibly suppresses of genes in that region expression.

The repressor elements used in the invention are preferably of mammalian origin, and most preferably human.

In addition to the repressor elements described above, functionally equivalent homologs of such elements and exhibiting extensive homology to the repressor elements present in human and other species, can be identified and readily isolated, without undue experimentation, by molecular biological techniques well known in the art. “Functionally equivalent”, as utilized herein, refers to a protein or polypeptide capable of exhibiting a substantially similar in vivo or in vitro activity as the respective repressor element

### 5.2. Construction of Modified ETS2 Gene Sequences

Described herein are methods for the construction of a gene construct encoding a modified ets2 gene product that can be expressed in cancer cells. Specifically described are the construction of a nucleotide sequence encoding a modified ets2, the insertion of the modified ets2 gene sequence into an appropriate cloning vector, and the introduction of the expression gene construct into the appropriate cells for production of antisense ets2 RNA, dominant negative mutant ets2 and ets2-repressor fusion proteins.

The procedures described in standard treatises, e.g., Methods in Enzymology, 1987, volume 154, Academic Press; Sambrook et al., 1989, Molecular Cloning-A Laboratory Manual, 2nd Edition, Cold Spring Harbor Press, New York; and Ausubel et al., Current Protocols in Molecular Biology, Greene Publishing Associates and Wiley Interscience, New York, may be followed to carry out routine molecular biology reactions used in constructing and modifying the ets2 gene construct. Methods described in detail infra are for illustration only and not by way of limitation. Various cloning vectors and expression systems that are commercially available may also be used according to the manufacturer's instructions.

### 5.2.1. Isolation of Ets2 Gene Sequences

In various aspects, the invention relates to amino acid sequences of modified ets2 proteins, and fragments and derivatives thereof, which are capable of binding to ets target sequences but inactive in initiating transcription of the ets-responsive gene associated with the ets target sequence. Nucleic acids encoding the modified ets2 described above are provided, as well as nucleic acids complementary to and capable of hybridizing to such nucleic acids.

Any eukaryotic cell potentially can serve as the nucleic acid source for obtaining the coding region of a ets2 gene. Nucleic acid sequences encoding ets2 can be isolated from vertebrate, mammalian, as well as primate sources, including humans.

The DNA may be obtained by standard procedures known in the art from cloned DNA (e.g., a DNA “library”), or by DNA amplification. Clones derived from genomic DNA may contain regulatory and intron DNA regions in addition to coding regions; clones derived from CDNA will contain only exon sequences. Whatever the source, the ets2 gene should be molecularly cloned into a suitable vector for propagation of the gene.

In the molecular cloning of a ets2 gene from genomic DNA, DNA fragments are generated and cloned to form a genomic library. Since some of the sequences encoding related ets2s are available and can be purified and labeled, the cloned DNA fragments in the genomic DNA library may be screened by nucleic acid hybridization to the labeled probe (Benton, W. and Davis, R., 1977, Science 196:180; Grunstein, M. And Hogness, D., 1975, Proc. Natl. Acad. Sci. U.S.A. 72:3961). Those DNA fragments with substantial homology to the probe will hybridize. It is also possible to identify the appropriate fragment by restriction enzyme digestion(s) and comparison of fragment sizes with those expected according to a known restriction map if such is available.

Alternatives to isolating the ets2 genomic DNA include, but are not limited to, chemically synthesizing the gene sequence itself from a known sequence or making cDNA to the mRNA which encodes the ets2. For example, RNA for cDNA cloning of the ets2 gene can be isolated from cells which express the ets2. A CDNA library may be generated by methods known in the art and screened by methods, such as those disclosed for screening a genomic DNA library. If an antibody to the ets2 is available, the ets2 may be identified by binding of labeled antibody to the putatively ets2 synthesizing clones.

Prior to modification, the ets2 gene can be inserted into an appropriate cloning vector and introduced into host cells so that many copies of the gene sequence are generated. A large number of vector-host systems known in the art may be used such as, but not limited to, bacteriophages such as lambda derivatives, or plasmids such as pBR322 or pUC plasmid derivatives or the Bluescript vector (Stratagene). Moreover, if the ets2 gene is inserted into an appropriate vector in the orientation opposite to normal transcription, antisense ets2 RNA molecules can be generated in quantities which can be used for direct injection into cancer cells.

The above methods are not meant to limit the methods by which clones of ets2 may be obtained or propagated. The modified ets2 of the invention are modified such that they can block the normal function of the wild type endogenous ets2.

In particular, a modified ets2 of the invention lacks a segment of the protein that activates transcription of a ets-responsive gene. The transcription function is disabled by deleting the segment, or by substitution with amino acid residues that render the segment non-functional. In a preferred embodiment, the activation domain of a ets2 is replaced by a repressor element.

### 5.2.2 Modification of Ets2 Genes

The modifications present in modified ets2 proteins and dominant negative mutants of ets 2 of the invention can be produced by various methods known in the art. The term modified ets2 gene or modified ets2 gene product as used herein encompasses dominant negative mutants of ets2 and nucleic acid molecules encoding therefor.

The manipulations which result in their production can occur at the gene or protein level, preferably at the gene level. For example, the cloned coding region of ets2 can be modified by any of numerous recombinant DNA methods known in the art (Sambrook et al., 1990, Molecular Cloning, A Laboratory Manual, 2d ed., Cold Spring Harbor Laboratory, Cold Spring Harbor, New York; Ausubel et al., in Chapter 8 of Current Protocols in Molecular Biology, Greene Publishing Associates and Wiley Interscience, New York). It will be apparent from the following discussion that substitutions, deletions, insertions, or any combination thereof are introduced or combined to arrive at a final nucleotide sequence encoding a modified ets2 protein.

Alternatively, modified ets2 can be chemically synthesized. For example, a peptide corresponding to a portion of a ets2 which comprises the desired modifications can be synthesized by use of a peptide synthesizer.

The activation domain of ets2 that is required for initiation of transcription of a ets-responsive gene but not necessary for binding to the ets target sequence can be disabled either by deleting the domain, or by obliterating the domain with non-conservative amino acid substitutions.

In order to remove the segment of DNA encoding the activation domain or other signals that facilitate transcription of an ets target gene, the ets2 gene sequence can be cleaved at appropriate sites with restriction endonuclease(s) if such sites are available, releasing a fragment of DNA encoding the domain. The remainder of the ets2 coding region is then isolated, and ligated to form the modified ets2 gene sequence.

Alternatively, if convenient restriction sites are not available, a larger fragment of DNA can be released by using restriction sites located in sequences flanking the region that encodes the activation domain, and replaced by a similar fragment of synthetic DNA which lacks the sequence encoding the domain. Care must be taken to ensure that the proper translation reading frame is maintained.

If it is desirable, restriction sites can be created in the appropriate positions by site-directed mutagenesis methods and/or DNA amplification methods known in the art. See, for example, Shankarappa et al., 1992, PCR Method Appl. 1:277-278. The polymerase chain reaction (PCR) is commonly used for introducing desired sequence changes into the DNA of interest. Any changes in primer sequence can be easily incorporated into the DNA product of PCR which facilitates subsequent incorporation of the changes into the gene sequence. For example, synthetic oligonucleotides incorporating the desired restriction site are used in conjunction with the appropriate flanking sequence primers to amplify two adjacent fragments of DNA. Each of these amplified fragments will contain the new restriction site at one end. Following enzymatic digestion at both the new and flanking sites, the amplified fragments are ligated and subcloned into a vector ready for further manipulations. It is imperative that the introduction of restriction sites does not alter the amino acid sequence of the encoded protein.

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson, C., et al., 1978, J. Biol. Chem 253:6551), oligonucleotide-directed mutagenesis (Smith, 1985, Ann. Rev. Genet. 19:423-463; Hill et al., 1987, Methods Enzymol. 155:558-568), PCR-based overlap extension (Ho et al., 1989, Gene 77:51-59), PCR-based megaprimer mutagenesis (Sarkar et al., 1990, Biotechniques, 8:404-407), etc. Modifications can be confirmed by DNA sequencing.

The above method can be applied to substitute one or more of the amino acid residues in the activation domain. Substitutes for an amino acid within the activation domain may be selected from members of a different class to which the amino acid belongs. The nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. The substitution which in general are expected to produce the greatest changes in biochemical properties will be those in which (a) a hydrophilic residue, e.g., scryl or threonyl, is substituted for (or by) a hydrophobic residue, e.g, leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g, phenylalanine, is substituted for (or by) one not having a side chain, e.g., glycine.

The above methods are not meant to limit the methods by which the activation domain and/or other amino acid residues important in facilitating transcription can be deleted or obliterated in ets2.

### 5.2.3 Ets2-repressor fusion

In another embodiment of the invention, modified ets2 protein includes a fusion protein comprising a modified ets2 gene product (including dominant negative mutant of ets2) and a transcription repressor element.

In various embodiments, such a fusion protein can be made by ligating a modified ets2 gene sequence to the sequence encoding the repressor element in the proper reading frame. If genomic sequences are used, care should be taken to ensure that the modified gene remains within the same translational reading frame, uninterrupted by translational stop signals and/or spurious messenger RNA splicing signals.

In a preferred embodiment, the repressor element is fused at its amino terminal to the carboxyl terminal of the ets2. The precise site at which the fusion is made in the carboxyl terminal is not critical. The optimal site can be determined by routine experimentation. The inhibitory activity of the modified ets2 can be tested by methods known in the art.

The repressor element in a variety of transcription repressor known in the art may be used in the modification of ets2, such as but not limited to those present in the proteins described in section 5.1.4, e.g., the Kruppel protein (the KRAB boxes), even-skipped, engrailed, v-erbA oncoprotein, thyroid hormone receptor, WT1 gene product, YY1, Gfi-1 oncogene (SNAG domain), etc.

Amino acid sequences and nucleotide sequences of naturally occurring transcription repressors are generally available in sequence databases, such as GenBank.

Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics.

Due to the degeneracy of the genetic code, the term “repressor gene sequence” refers not only to the naturally occurring nucleotide sequence but also encompasses all the other degenerate DNA sequences that encode a repressor element. As will be appreciated by those skilled in the art, many methods can be used to obtain the coding region of the above-mentioned transcription repressors, including but not limited to, DNA cloning, DNA amplification, and synthetic methods.

Other specific embodiments for the cloning of a nucleotide sequence encoding a repressor, are presented as examples but not by way of limitation, as follows:

In a specific embodiment, nucleotide sequences encoding transcription repressors within a family can be identified and obtained by hybridization with a probe comprising nucleotide sequence encoding a repressor element under conditions of low to medium stringency.

By way of example and not limitation, procedures using such conditions of low stringency are as follows (see also Shilo and Weinberg, 1981, Proc. Natl. Acad. Sci. USA 78:6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

In another embodiment, polymerase chain reaction (PCR) is used to amplify the desired sequence in DNA clone or a genomic or cDNA library, prior to selection. PCR can be carried out, e.g., by use of a thermal cycler and Taq polymerase (Gene Amp™). The DNA being amplified can include CDNA or genomic DNA from any species. One can choose to synthesize several different degenerate primers, for use in the PCR reactions. After successful amplification, the sequence encoding a repressor element may be cloned and sequenced.

### 5.3 Production of Modified Ets2 Gene Product

In various embodiments of the invention, sequences encoding modified ets2 gene product, including dominant negative mutants of ets2 and ets2-repressor fusion protein, are inserted into an expression vector for propagation and expression in recombinant cells, and for introduction into cancer cells.

In specific embodiments, a lentiviral vector can be used to express ets2 or modified gene product (Case et al., 1999, Proc. Natl. Acad. Sci. 96: 2988-2993; Miyoshi et al., 1998, J. Virology 72: 8150-8157).

An expression construct, as used herein, refers to a nucleotide sequence encoding a modified ets2 operably associated with one or more regulatory regions which enables expression of the modified ets2 in an appropriate host cell. “Operably-associated” refers to an association in which the regulatory regions and the modified ets2 sequence to be expressed are joined and positioned in such a way as to permit transcription, and ultimately, translation.

Such expression construct can also be used to produce antisense ets2 RNA, provided that the nucleotide sequence encoding ets2 is inserted in the reverse orientation.

The regulatory regions necessary for transcription of the modified ets2 can be provided by the expression vector. A translation initiation codon (ATG) may also be provided if the modified ets2 sequence lacking its cognate initiation codon is to be expressed. In a compatible host-expression construct system, cellular transcriptional factors, such as RNA polymerase, will bind to the regulatory regions on the expression construct to effect transcription of the modified ets2 sequence in the host cell. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′-non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

Both constitutive and inducible regulatory regions may be used for expression of the modified ets2. It may be desirable to use inducible promoters to control the high level expression of the modified ets2 once the expression construct is introduced into cancer cells in vivo. Examples of useful regulatory regions are provided in the next section below.

In order to attach DNA sequences with regulatory functions, such as promoters, to the modified ets2 gene sequence or to insert the modified ets2 gene sequence into the cloning site of a vector, linkers or adapters providing the appropriate compatible restriction sites may be ligated to the ends of the cDNAs by techniques well known in the art (Wu et al., 1987, Methods in Enzymol 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising a modified ets2 sequence operably associated with regulatory regions can be directly introduced into appropriate host cells for expression and production of modified ets2 protein without further cloning. See, for example, U.S. Pat. No. 5,580,859. The expression constructs can also contain DNA sequences that facilitate integration of the modified ets2 sequence into the genome of the host cell, e.g., via homologous recombination. In this instance, it is not necessary to employ an expression vector comprising a replication origin suitable for appropriate host cells in order to propagate and express the modified ets2 in the host cells.

### 5.3.1 Host-Vector Systems

Described herein are systems of vectors and host cells that can be used for the expression of modified ets2s. A variety of expression vectors may be used in the present invention which include, but are not limited to, plasmids, cosmids, phage, phagemids, or modified viruses. Typically, such expression vectors comprise a functional origin of replication for propagation of the vector in an appropriate host cell, one or more restriction endonuclease sites for insertion of the modified ets2 gene sequence, and one or more selection markers. The expression vector must be used with a compatible host cell which may be derived from a prokaryotic or an eukaryotic organism including but not limited to bacteria, yeasts, insects, mammals, and humans.

In one aspect, expression constructs and vectors are introduced into host cells for the purpose of producing the modified ets2. Any cell type that can produce mammalian proteins and is compatible with the expression vector may be used, including those that have been cultured in vitro or genetically engineered. Host cells may be obtained from normal or affected subjects, including healthy humans, cancer patients, and patients infected with a virus, private laboratory deposits, public culture collections such as the American Type Culture Collection, or from commercial suppliers.

In another aspect, expression constructs are introduced into cancer cells for the purpose of gene therapy. Cells into which a modified ets2 gene sequence can be introduced for purposes of production of the modified ets2 in vivo may include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. For instance, the lentiviral vector was used for transduction of quiescent, primitive human hematopoietic progenitor cells and may provide therapeutically useful levels of gene transfer into human hematopoietic stem cells (Case et al., 1999, Proc. Natl. Acad. Sci. USA 96: p.2988-2993). The choice of cell type depends on the type of tumor being treated or prevented, and can be determined by one of skill in the art.

Different host cells have characteristic and specific mechanisms for the posttranslational processing and modification of proteins. A host cell may be chosen which modifies and processes the expressed gene products in a specific fashion similar to the way the recipient processes its ets2. For the purpose of producing large amounts of modified ets2 for adminstration to a subject, it is preferable that the type of host cell used in the present invention has been used for expression of heterologous genes, and is reasonably well characterized and developed for large-scale production processes.

In a particular embodiment, an expression construct comprising a modified ets2 gene sequence is introduced into a preneoplastic or neoplastic cell.

Vectors based on E. coli are the most popular and versatile systems for high level expression of foreign proteins (Makrides, 1996, Microbiol Rev, 60:512-538). Nonlimiting examples of regulatory regions that can be used for expression in E. coli may include but not limited to lac, trp, lpp, phoA, recA, tac, T3, T7 and λPL (Makrides, 1996, Microbiol Rev, 60:512-538). Non-limiting examples of prokaryotic expression vectors may include the λgt vector series such as λgt11 (Huynh et al., 1984 in “DNA Cloning Techniques”, Vol. I: A Practical Approach (D. Glover, ed.), pp. 49-78, IRL Press, Oxford), and the pET vector series (Studier et al., 1990, Methods Enzymol., 185:60-89). However, a potential drawback of a prokaryotic host-vector system is the inability to perform many of the post-translational processing of mammalian cells. Thus, an eukaryotic host-vector system is preferred, a mammalian host-vector system is more preferred, and a human hostvector system is the most preferred.

For expression of modified ets2 in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein gene, mouse mammary tumor virus glucocorticoid responsive long terminal repeats (MMTV-LTR), β-interferon gene, and hsp70 gene (Williams et al., 1989, Cancer Res. 49:2735-42; Taylor et al., 1990, Mol. Cell Biol., 10:165-75).

The following animal regulatory regions, which exhibit tissue specificity and have been utilized in transgenic animals, can also be used in tumor cells of a particular tissue type: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); gonadotropic releasing hormone gene control region which is active in the hypothalamus (Mason et al., 1986, Science 234:1372-1378); probasin and prostate-specific antigen (PSA) gene control region which is active in the prostate (Brookes et al., 1998, The Prostate 35:18-26).

The efficiency of expression of the modified ets2 in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153:516-544; Gorman, 1990, Curr. Op. in Biotechnol. 1:36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors which can be replicated and maintained in at least two types of host cells.

In addition, the expression vector may contain selectable or screenable marker genes for initially isolating, identifying or tracking host cells that contain DNA encoding a modified ets2. For long term, high yield production of modified ets2-peptide complexes, stable expression in mammalian cells is preferred. A number of selection systems may be used for mammalian cells, including but not limited to the Herpes simplex virus thymidine kinase (Wigler et al., 1977, Cell 11:223), hypoxanthine-guanine phosphoribosyltransferase (Szybalski and Szybalski, 1962, Proc. Natl. Acad. Sci. USA 48:2026), and adenine phosphoribosyltransferase (Lowy et al., 1980, Cell 22:817) genes can be employed in tk, hgprt or aprt cells, respectively. Also, antimetabolite resistance can be used as the basis of selection for dihydrofolate reductase (dhfr), which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neomycin phosphotransferase (neo), which confers resistance to the aminoglycoside G-418 (Colberre-Garapin et al., 1981, J. Mol. Biol. 150:1); and hygromycin phosphotransferase (hyg), which confers resistance to hygromycin (Santerre et al., 1984, Gene 30:147). Other selectable markers, such as but not limited to histidinol and Zeocin™ can also be used.

Preferred mammalian host cells include but are not limited to those derived from humans, monkeys and rodents, (see, for example, Kriegler M. in “Gene Transfer and Expression: A Laboratory Manual”, New York, Freeman & Co. 1990).

A number of viral-based expression systems may also be utilized with mammalian cells to produce modified ets2s. Vectors using DNA virus backbones have been derived from simian virus 40 (SV40) (Hamer et al., 1979, Cell 17:725), adenovirus (Van Doren et al., 1984, Mol Cell Biol 4:1653), adeno-associated virus (McLaughlin et al., 1988, J Virol 62:1963), and bovine papillomas virus (Zinn et al., 1982, Proc Natl Acad Sci 79:4897). In cases where an adenovirus is used as an expression vector, the donor DNA sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts. (See e.g., Logan and Shenk, 1984, Proc. Natl. Acad. Sci. (USA) 81:3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene construct are transfected into cultured mammalian cells by, for example, the calcium phosphate coprecipitation technique. For those host cells that do not manifest a transformed phenotype, selection of transformants is achieved by use of a dominant selectable marker, such as histidinol and G418 resistance. For example, a modified ets2 gene sequence can be inserted into BPV vectors, such as pBCMGSNeo and pBCMGHis (Karasuyama et al., Eur. J. Immunol. 18:97-104; Ohe et al., Human Gene Therapy, 6:325-33) which can then be transfected into a diverse range of cell types for expression of the modified ets2.

Alternatively, the vaccinia 7.5K promoter may be used. (See, e.g., Mackett et al., 1982, Proc. Natl. Acad. Sci. (USA) 79:7415-7419; Mackett et al., 1984, J. Virol. 49:857-864; Panicali et al., 1982, Proc. Natl. Acad. Sci. 79:4927-4931.) In cases where a human host cell is used, vectors based on the Epstein-Barr virus (EBV) origin (OriP) and EBV nuclear antigen 1 (EBNA-1; a trans-acting replication factor) can be used. Such vectors can be used with a broad range of human host cells, e.g., EBO-pCD (Spickofsky et al., 1990, DNA Prot Eng Tech 2:14-18); pDR2 and λDR2 (available from Clontech Laboratories).

Modified ets2 may also be made with a retrovirus-based expression system. Retroviruses, such as Moloney murine leukemia virus, can be used since most of the viral gene sequence can be removed and replaced with modified ets2 gene sequence while the missing viral functions can be supplied in trans. In contrast to transfection, retroviruses can efficiently infect and transfer genes to a wide range of cell types including, for example, primary hematopoietic cells. Moreover, the host range for infection by a retroviral vector can be manipulated by the choice of envelope used for vector packaging.

For example, a retroviral vector can comprise a 5′ long terminal repeat (LTR), a 3′ LTR, a packaging signal, a bacterial origin of replication, and a selectable marker. The modified ets2 DNA is inserted into a position between the 5′ LTR and 3′ LTR, such that transcription from the 5′ LTR promoter transcribes the cloned DNA. The 5′ LTR comprises a promoter, including but not limited to an LTR promoter, an R region, a U5 region and a primer binding site, in that order. Nucleotide sequences of these LTR elements are well known in the art. A heterologous promoter as well as multiple drug selection markers may also be included in the expression vector to facilitate selection of infected cells. See, McLauchlin et al., 1990, Prog Nucleic Acid Res and Molec Biol 38:91-135; Morgenstern et al., 1990, Nucleic Acid Res 18:3587-3596; Choulika et al., 1996, J Virol 70:1792-1798; Boesen et al., 1994, Biotherapy 6:291-302; Salmons and Gunzberg, 1993, Human Gene Therapy 4:129-141; and Grossman and Wilson,1993, Curr. Opin. in Genetics and Devel. 3:110-114.

Other useful eukaryotic host-vector system may include yeast and insect systems. In yeast, a number of vectors containing constitutive or inducible promoters may be used with Saccharomyces cerevisiae (baker's yeast), Schizosaccharomyces pombe (fission yeast), Pichia pastoris, and Hansenula polymorpha (methylotropic yeasts). For a review see, Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathem et al., Cold Spring Harbor Press, Vols. I and II.

In an insect system, Autographa californica nuclear polyhidrosis virus (AcNPV) a baculovirus, can be used as a vector to express modified ets2 in Spodoptera frugiperda cells. The modified ets2 gene sequences may be cloned into non-essential regions (for example the polyhedrin gene) of the virus and placed under control of an AcNPV promoter (for example the polyhedrin promoter). These recombinant viruses are then used to infect host cells in which the inserted DNA is expressed. (See e.g., Smith et al., 1983, J Virol 46:584; Smith, U.S. Pat. No. 4,215,051.)

Any of the cloning and expression vectors described herein may be synthesized and assembled from known DNA sequences by well known techniques in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

The resulting recombinant modified ets2 can be delivered to the cells in a patient by various methods known in the art.

### 5.3.2. Expression of Modified Ets2

Expression constructs containing cloned nucleotide sequence encoding modified ets2 can be introduced into the host cell by a variety of techniques known in the art, including but not limited to, for prokaryotic cells, bacterial transformation (Hanahan, 1985, in DNA Cloning, A Practical Approach, 1:109-136), and for eukaryotic cells, calcium phosphate mediated transfection (Wigler et al., 1977, Cell 11:223-232), liposome-mediated transfection (Schaefer-Ridder et al., 1982, Science 215:166-168), electroporation (Wolff et al., 1987, Proc Natl Acad Sci 84:3344), and microinjection (Cappechi, 1980, Cell 22:479488).

For long term, high yield production of properly processed modified ets2, expression in mammalian cells is preferred. Cell lines that stably express modified ets2 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while modified ets2 is expressed continuously.

The recombinant cells may be cultured under standard conditions of temperature, incubation time, optical density, and media composition.

In an embodiment where the expression construct comprising sequences encoding a modified ets2 is used in gene therapy, the modified ets2 gene sequence is introduced into cancer cells in vivo. Such introduction can be carried out by any method known in the art, such as but not limited to transfection, transduction, microinjection, infection with a viral or bacteriophage vector containing the modified ets2 gene sequences, liposome-mediated gene transfer, microcell-mediated gene transfer, etc. Numerous techniques are known in the art for the introduction of foreign genes into cells (see e.g., Loeffler and Behr,1993, Meth. Enzymol. 217:599-618; Cohen et al.,1993, Meth. Enzymol. 217:618-644; Cline, 1985, Pharmac. Ther. 29:69-92) may be used in accordance with the present invention, provided that the physiological functions of the recipient are not disrupted. The technique should provide for the stable transfer of the modified ets2 gene sequence to the cell, so that the sequence is expressible by the cell and preferably heritable and expressible by its cell progeny.

### 5.4 Antibodies to Ets2 Gene Products

In another embodiment, the present invention relates to the uses of antibodies or fragments thereof capable of specifically recognizing one or more epitopes of the ets2 gene products, epitopes of conserved variants of the ets2 gene products, epitopes of mutant ets2 gene products, or peptide fragments of the ets2 gene products. Such antibodies may include, but are not limited to, polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above.

Such antibodies may be used, for example, in the detection of a ets2 gene product in an biological sample and may, therefore, be utilized as part of a diagnostic or prognostic technique whereby patients may be tested for abnormal levels of ets2 gene products, and/or for the presence of abnormal forms of the such gene products. Such antibodies may also be included as a reagent in a kit for use in a diagnostic or prognostic technique. Such antibodies may also be utilized in conjunction with, for example, compound screening schemes, as described, below, for the evaluation of the effect of test compounds on ets2 gene product levels and/or activity. Antibodies to ets2 gene product may be used in a method for the inhibition of abnormal ets2 gene product activity. Thus, such antibodies may, therefore, be utilized as part of cancer treatment methods. Additionally, such antibodies can be used in conjunction with the gene therapy techniques described, below, to, for example, evaluate the normal and/or engineered ets2-expressing cells prior to their introduction into the patient.

Described herein are methods for the production of antibodies of such antibodies or fragments thereof. Any of such antibodies or fragments thereof may be produced by standard immunological methods or by recombinant expression of nucleic acid molecules encoding the antibody or fragments thereof in an appropriate host organism.

For the production of antibodies against a ets2 gene product, various host animals may be immunized by injection with a ets2 gene product, or a fragment thereof. Fragments of ets2 can be synthesized as antigenic peptides in accordance with the known amino acid sequence of ets2. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a ets2 gene product, or an antigenic functional derivative thereof. For the production of polyclonal antibodies, host animals such as those described above, may be immunized by injection with ets2 gene product supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against ets2 gene products. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., 1988, Science 242:103825 -1041).

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

### 5.5 Methods of Diagnosis of Cancer

In one embodiment, the present invention provides a variety of methods for the diagnostic and prognostic evaluation of cancer. Such methods may, for example, utilize reagents such as the ets2 gene nucleotide sequences, and antibodies directed against ets2 gene products, including peptide fragments thereof, as described, above.

Specifically, such reagents may be used, for example, for: (1) the detection of the presence of ets2 gene mutations, or the detection of either over- or under-expression of ets2 gene mRNA in preneoplastic or neoplastic cells relative to normal cells, or the qualitative or quantitative detection of other alleic forms of ets2 transcripts which may correlate with cancer or susceptibility toward neoplastic changes, and (2) the detection of an over-abundance of ets2 gene product relative to the non-disease state or the presence of a modified (e.g., less than full length) ets2 gene product which correlates with a neoplastic state or a progression toward neoplasia or metastasis.

The methods described herein may be applied to samples of cells or cellular materials taken directly from a patient. Any method known in the-art for collection or isolation of the desired cells or materials can be used. In particular, for prostate cancer, samples for testing may be obtained by techniques known in the art, such as percutaneous fine needle aspiration biopsy with endoscopic ultrasonography.

The methods described herein may be performed, for example, by utilizing pre-packaged diagnostic test kits comprising at least one specific ets2 gene nucleic acid or anti-ets2 gene antibody reagent described herein, which may be conveniently used, e.g., in clinical settings or in home settings, to diagnose patients exhibiting preneoplastic or neoplastic abnormalities, and to screen and identify those individuals exhibiting a predisposition to such neoplastic changes.

The present invention is useful for the diagnosis and prognosis of malignant diseases in which the ets2 gene or gene product is implicated or is suspected to be implicated. Such malignancies include but are not limited to cancer of the liver, ovary, breast, lung, bladder, kidney, colon, rectum, prostate gland and cervix.

### 5.5.1 Detection of Ets2 Gene Nucleic Acid Molecules

Quantitative and qualitative aspects of ets2 gene expression can also be assayed. Nucleic acid from any nucleated cell can be used as the starting point for such assay techniques, and may be isolated according to standard nucleic acid preparation procedures which are well known to those of skill in the art. For the detection of ets2 mutations, any nucleated cell can be used as a starting source for genomic nucleic acid. For the detection of ets2 transcripts or ets2 gene products, any cell type or tissue in which the ets2 gene is expressed, such as, for example, prostate cancer cells, including metastases, may be utilized.

Diagnostic methods for the detection of ets2 gene specific nucleic acid molecules, in patient samples (such as prostate juice or serum) or other appropriate cell sources, may involve the amplification of specific gene sequences, e.g., by the polymerase chain reaction (PCR; see Mullis, K. B., 1987, U.S. Pat. No. 4,683,202), followed by the analysis of the amplified molecules using techniques well known to those of skill in the art.

The isolated cells can be derived from cell culture or from a patient. The analysis of cells taken from culture may be a necessary step in the assessment of cells to be used as part of a cell-based gene therapy technique or, alternatively, to test the effect of compounds on the expression of the ets2 gene. Such analyses may reveal both quantitative and qualitative aspects of the expression pattern of the ets2 gene, including activation or inactivation of ets2 gene expression and presence of mutations.

In one embodiment of such a detection scheme, a CDNA molecule is synthesized from an RNA molecule of interest by reverse transcription. All or part of the resulting CDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among the ets2 gene nucleic acid reagents described in Section 5.1. The preferred lengths of such nucleic acid reagents are at least 9-30 nucleotides.

For detection of the amplified product, the nucleic acid amplification may be performed using radioactively or non-radioactively labeled nucleotides. In some cases, enough amplified product may be made such that the product may be visualized by standard ethidium bromide staining or by utilizing any other suitable nucleic acid staining method.

Such RT-PCR techniques can be utilized to detect differences in ets2 transcript size which may be due to normal or abnormal alternative splicing. Additionally, such techniques can be performed using standard techniques to detect quantitative differences between levels of full length and/or alternatively spliced ets2 transcripts detected in normal individuals relative to those individuals having cancer or exhibiting a predisposition toward neoplastic changes.

In the case where detection of specific alternatively spliced species or mutants is desired, appropriate primers and/or hybridization probes can be used, such that, in the absence of such sequence, no amplification would occur. Alternatively, primer pairs may be chosen utilizing the sequence data depicted in FIG. 6 to choose primers which will yield fragments of differing size depending on whether a particular exon is present or absent from the ets2 transcript, or the choice of polyA signal being utilized.

As an alternative to amplification techniques, standard Northern analyses can be performed if a sufficient quantity of the appropriate cells can be obtained. Utilizing such techniques, quantitative as well as size related differences between ets2 transcripts can also be detected.

Additionally, it is possible to perform such ets2 gene expression assays “in situ”, i.e., directly upon tissue sections (fixed and/or frozen) of patient tissue obtained from biopsies or resections, such that no nucleic acid purification is necessary. Nucleic acid reagents such as those described in Section 5.1 may be used as probes and/or primers for such in situ procedures (see, for example, Nuovo, G. J., 1992, “PCR In Situ Hybridization: Protocols And Applications”, Raven Press, N.Y.).

The results obtained by the methods described herein may be combined with diagnostic test results based on other genes that are also implicated in the pathology of the cancer. For example, K-ras and p53 mutations are often observed in patients.

### 5.5.2 Detection of Ets2 Gene Products

Antibodies directed against wild type or mutant ets2 gene products or conserved variants or peptide fragments thereof, which are discussed, above, in Section 5.4, may also be used as diagnostics and prognostics, as described herein. Such diagnostic methods, may be used to detect abnormalities in the level of ets2 gene expression, or abnormalities in the structure and/or temporal, tissue, cellular, or subcellular location of ets2 gene product.

The tissue or cell type to be analyzed will generally include those which are known, or suspected, to express the ets2 gene, such as, for example, prostate cancer cells or metastatic cells. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, New York), which is incorporated herein by reference in its entirety. The isolated cells can be derived from cell culture or from a patient. The analysis of cell taken from culture may be a necessary step to test the effect of compounds on the expression of the ets2 gene.

Preferred diagnostic methods for the detection of ets2 gene products or conserved variants or peptide fragments thereof, may involve, for example, immunoassays wherein the ets2 gene products or conserved variants, including gene products which are the result of alternatively spliced transcripts, or peptide fragments are detected by their interaction with an anti-ets2 gene product-specific antibody.

For example, antibodies, or fragments of antibodies, such as those described, above, in Section 5.4, useful in the present invention may be used to quantitatively or qualitatively detect the presence of ets2 gene products or conserved variants or peptide fragments thereof. The antibodies (or fragments thereof) useful in the present invention may, additionally, be employed histologically, as in immunofluorescence or immunoelectron microscopy, for in situ detection of ets2 gene products or conserved variants or peptide fragments thereof. In situ detection may be accomplished by removing a histological specimen from a patient, such as paraffin embedded sections of breast tissues and applying thereto a labeled antibody of the present invention. The antibody (or fragment) is preferably applied by overlaying the labeled antibody (or fragment) onto a biological sample. It may also be desirable to introduce the antibody inside the cell, for example, by making the cell membrane permeable. Through the use of such a procedure, it is possible to determine not only the presence of the ets2 gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for ets2 gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying ets2 gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled ets2 gene specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-ets2 gene product antibody may be determined according to well known methods. Those skilled in the art will be able to determine operative and optimal assay conditions for each determination by employing routine experimentation.

In various embodiments, the present invention provides the measurement of ets2 gene products, and the uses of such measurements in clinical applications.

The measurement of ets2 gene product of the invention can be valuable in detecting and/or staging cancer in a subject, in screening of cancer in a population, in differential diagnosis of the physiological condition of a subject, and in monitoring the effect of a therapeutic treatment on a subject.

The present invention also provides for the detecting, diagnosing, or staging of cancer, or the monitoring of treatment of cancer by measuring in addition to ets2 gene product at least one other marker, such as receptors or differentiation antigens. For example, serum markers selected from, for example but not limited to, carcinoembryonic antigen (CEA), and prostate specific antigen (PSA) can be measured in combination with ets2 gene product to detect, diagnose, stage, or monitor treatment of prostate cancer. In another embodiment, the prognostic indicator is the observed change in different marker levels relative to one another, rather than the absolute levels of the markers present at any one time. These measurements can also aid in predicting therapeutic outcome and in evaluating and monitoring the overall disease status of a subject.

### 5.5.3 Monitoring the Effect of a Therapeutic Treatment

The present invention provides a method for monitoring the effect of a therapeutic treatment on a subject who has undergone the therapeutic treatment.

Clinicians very much need a procedure that can be used to monitor the efficacy of these treatments. Ets2 gene product can be identified and detected in cancer patients with different manifestations of disease, providing a sensitive assay to monitor therapy. The therapeutic treatments which may be evaluated according to the present invention include but are not limited to radiotherapy, surgery, chemotherapy, vaccine administration, endocrine therapy, immunotherapy, and gene therapy, etc. The chemotherapeutic regimens include, but are not limited to administration of drugs such as, for example, fluorouracil and taxol.

The method of the invention comprises measuring at suitable time intervals before, during, or after therapy, the amount of a ets2 gene product. Any change or absence of change in the amount of the ets2 gene product can be identified and correlated with the effect of the treatment on the subject, such as, for example, a reduction of the transformed phenotype in cancer cells.

In a preferred aspect, the approach that can be taken is to determine the levels of ets2 gene product levels at different time points and to compare these values with a baseline level. The baseline level can be either the level of the marker present in normal, disease free individuals; and/or the levels present prior to treatment, or during remission of disease, or during periods of stability. These levels can then be correlated with the disease course or treatment outcome. Elevated levels of ets2 gene product relative to the baseline level indicate a poor response to treatment.

### 5.6 Screening Assays for compounds that Modulate Ets2 Activity

The present invention further provides methods for the identification of compounds that may, through its interaction with the ets2 gene or ets2 gene product, affect the onset, progression and metastatic spread of cancer; especially prostate cancer.

The following assays are designed to identify: (i) compounds that bind to ets2 gene products, including mammalian and non-mammalian homologs of ets2; (ii) compounds that bind to other intracellular proteins and/or segments of nucleic acid that interact with a ets2 gene product, including mammalian and non-mammalian homologs of ets2; (iii) compounds that interfere with the interaction of the ets2 gene product, including mammalian and non-mammalian homologs of ets2, with other intracellular proteins and/or segments of nucleic acid; and (iv) compounds that modulate the activity of ets2 gene (i.e., modulate the level of ets2 gene expression and/or modulate the level of ets2 gene product activity).

Assays may additionally be utilized which identify compounds which bind to ets2 gene regulatory sequences (e.g., promoter sequences). See e.g., Platt, 1994, J. Biol. Chem. 269:28558-28562, which is incorporated herein by reference in its entirety. Also provided is a method for identifying compounds that modulate ets2 gene expression, comprising: (a) contacting a test compound with a cell or cell lysate containing a reporter gene operatively associated with a ets2 gene regulatory element; and (b)detecting expression of the reporter gene product. Also provided is another method for identifying compounds that modulate ets2 gene expression comprising: (a) contacting a test compound with a cell or cell lysate containing ets2 transcripts; and (b) detecting the translation of the ets2 transcript. Any reporter gene known in the art can be used, such as but limited to, green fluorescent protein, β-galactosidase, alkaline phosphatase, chloramphenicol acetyltransferase, etc.

### 5.6.1 In Vitro Screening Assays for Compounds that Bind to the ets2 Gene Product

In vitro systems may be designed to identify compounds capable of interacting with, e.g., binding to, the ets2 gene products of the invention and homologs of ets2. Compounds identified may be useful, for example, in modulating the activity of wild type and/or mutant ets2 gene products, may be useful in elaborating the biological function of the ets2 gene product, may be utilized in screens for identifying compounds that disrupt normal ets2 gene product interactions, or may in themselves disrupt such interactions.

The principle of the assays used to identify compounds that interact with the ets2 gene product involves preparing a reaction mixture of the ets2 gene product, or fragments thereof and the test compound under conditions and for a time sufficient to allow the two components to interact with, e.g., bind to, thus forming a complex, which can represent a transient complex, which can be removed and/or detected in the reaction mixture. These assays can be conducted in a variety of ways. For example, one method to conduct such an assay would involve anchoring ets2 gene product or the test substance onto a solid phase and detecting ets2 gene product/test compound complexes anchored on the solid phase at the end of the reaction. In one embodiment of such a method, the ets2 gene product or fragment thereof may be anchored onto a solid surface, and the test compound, which is not anchored, may be labeled, either directly or indirectly.

In practice, microtitre plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for ets2 gene product or the test compound to anchor any complexes formed in solution, and a labeled antibody specific for the other component of the possible complex to detect anchored complexes.

### 5.6.2 Assays for Intracellular Proteins that Interact with the ets2 Gene Product

Any method suitable for detecting protein-protein interactions or proteinnucleic acid interactions may be employed for identifying ets2 protein-intracellular protein interactions, especially interactions mediated by the ets2 domain.

Among the traditional methods which may be employed are co-immunoprecipitation, crosslinking and co-purification through gradients or chromatographic columns. Surface display library and yeast-based two-hybrid system can also be utilized to isolate the gene encoding such ets2-binding proteins.

These methods allows the identification of molecules, including intracellular proteins, that interact with ets2 gene products. Once isolated, such a protein can be sequenced using techniques well-known to those of skill in the art, such as by Edman degradation (see, e.g., Creighton, 1983, “Proteins: Structures and Molecular Principles,” W. H. Freeman & Co., N.Y., pp.34-49). The amino acid sequence obtained may be used as a guide for the generation of oligonucleotide mixtures that can be used to screen for gene sequences encoding such proteins. Screening made be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Another method that detects protein interactions in vivo is the two-hybrid system, which is described here for illustration only and not by way of limitation. One example of this approach has been described (Chien, et al., 1991, Proc. Natl. Acad. Sci. USA, 88:9578-9582) and a kit is commercially available from Clontech (Palo Alto, Calif.).

The two-hybrid system or related methodologies may be used to screen activation domain libraries for proteins that interact with a “bait” gene product. By way of example, and not by way of limitation, ets2 gene products may be used as the bait gene product. Total genomic or cDNA sequences are fused to the DNA encoding an activation domain. This library and a plasmid encoding a hybrid of a bait ets2 gene product fused to the DNA-binding domain are co-transformed into a yeast reporter strain, and the resulting transformants are screened for those that express the reporter gene. For example, a bait ets2 gene sequence, such as the open reading frame of the ets2 gene, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A c DNA library of the cell line from which proteins that interact with bait ets2 gene product are to be detected can be made using methods routinely practiced in the art. According to the particular system described herein, for example, the CDNA fragments can be inserted into a vector such that they are translationally fused to the transcriptional activation domain of GAL4. Such a library can be co-transformed along with the bait ets2 gene-GAL4 fusion plasmid into a yeast strain that contains a lacZ gene driven by a promoter that contains GAL4 activation sequence. A cDNA encoded protein, fused to a GAL4 transcriptional activation domain that interacts with bait ets2 gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait ets2 gene product-interacting protein using techniques routinely practiced in the art.

Additionally, methods may be employed which result in the identification of nucleic acids which interacts with the ets2 protein, i.e., ets2 target sites. These methods include, for example, probing gene libraries with labeled ets2 protein or fragments thereof (e.g., ets2 domain), using ets2 protein or fragments thereof in a manner similar to the well-known technique of antibody probing of λgt11 libraries. Such methods can also be adapted to monitor the interactions of dominant negative mutants of ets2 and ets2 target sites. Furthermore, novel ets2 target sequences can be isolated by the RNA differential display technique and whole genome PCR. See Robinson et al., 1997, Proc. Natl. Acad. Sci. USA, 94:7170-7175.

### 5.6.3 Assays for Compounds that Interfere with Ets2 Gene Product/Intracellular Macromolecular Interaction

The ets2 gene products of the invention, fragments thereof, and homologs of ets2 may, in vivo, interact with one or more intracellular macromolecules, such as proteins and nucleic acid molecules. Such macromolecules may include, but are not limited to DNA, RNA (including polyadenylated (poly(A)) RNA and RNA with the 5′ cap structure) and those proteins identified via methods such as those described, above, in Section 5.6.2. For purposes of this discussion, such intracellular macromolecules are referred to herein as “interacting partners”. Compounds that disrupt ets2 interactions in this way may be useful in regulating the activity of the ets2 gene product, including mutant ets2 gene products. Such compounds may include, but are not limited to molecules such as peptides, and the like, which would be capable of gaining access to the intracellular ets2 gene product.

The basic principle of the assay systems used to identify compounds that interfere with the interaction between the ets2 gene product and its intracellular interacting partner or partners involves preparing a reaction mixture containing the ets2 gene product, or fragments thereof, and the interacting partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of ets2 gene product and its intracellular interacting partner. Control reaction mixtures are incubated without the test compound or with a vehicle or carrier. The formation of any complexes between the ets2 gene product or fragments thereof and the intracellular interacting partner is then detected. The formation of a complex in the control reaction, but not in the reaction mixture containing the test compound, indicates that the compound interferes with the interaction of the ets2 gene protein and the interacting partner. Additionally, complex formation within reaction mixtures containing the test compound and normal ets2 gene protein may also be compared to complex formation within reaction mixtures containing the test compound and a mutant ets2 gene protein. This comparison may be important in those cases wherein it is desirable to identify compounds that disrupt interactions of mutant but not normal ets2 gene proteins.

### 5.6.4 Cell-Based Assays for Identification of Compounds which Modulate Ets2 Activity

Cell-based methods are presented herein which identify compounds capable of treating cancer by modulating ets2 activity. Specifically, such assays identify compounds which affect ets2-dependent processes, such as but not limited to changes in cell morphology, cell division, differentiation, adhesion, motility, or tumorigenicity.

In another embodiment, the cell-based assays are based on expression of the ets2 gene product in a mammalian cell and measuring the ets2-dependent process. Any mammalian cells that can express the ets2 gene and allow the functioning of the ets2 gene product can be used, in particular, cancer cells derived from the prostate gland. Other cancer cell lines such as those derived from prostate, liver, ovary, breast, lung, rectum, kidney and non-erythroid hemopoietic cells, may also be used provided that a detectable ets2 gene product is produced. Recombinant expression of the ets2 gene in these cells or other normal cells can be achieved by methods described above. In these assays, cells producing functional ets2 gene products are exposed to a test compound for an interval sufficient for the compound to modulate the activity of the ets2 gene product. The activity of ets2 gene product can be measured directly or indirectly through the detection or measurement of ets2-dependent cellular processes such as, for example, the manifestation of a transformed phenotype. As a control, a cell not producing the ets2 gene product may be used for comparisons. Depending on the cellular process, any techniques known in the art may be applied to detect or measure it.

### 5.6.5 Tumorigenicity Tests

The methods of the present invention further include observing for a reduction in tumorigenicity, i.e. tumorigenic potential, of the sample tumor cells. This step includes observing for an inhibition of or reduction in sample tumor cell growth. Measures of tumor cell growth that are suitable include, for example, growth rate, colony formation in soft agar, tumorigenicity in an experimental animal, tumor cell phenotype, thymidine incorporation, and drug sensitivity. All of these are factors which alone or in combination with one another can be used to determine whether or not cell tumorigenicity has been reduced. A “reduction in tumorigenicity” is said to have occurred when, for example, a reduction, i.e. inhibition, in growth rate, colony formation in soft agar, tumor size, thymidine incorporation, etc. is observed. Additionally, it will be readily apparent to those of ordinary skill in the art that tumor phenotype(s), as noted by the clinician or other qualified observer, can be used to determine whether there has been a reduction in cell tumorigenicity. Observation for inhibition of growth is usually made daily, although other time intervals are practiced.

The growth rate, for example, can be measured by macroscopically observing how rapidly the tumor cells grow. This may be expressed as a doubling time (the amount of time it takes for the cells to double their numbers). Tumor cell growth rate can be measured in tissue culture by adding a fixed number of cells in tissue culture medium to a flask, culturing them in a 5% CO₂ humidified atmosphere, removing and counting an aliquot of the cells at different time points. By plotting the cell counts over time, it is possible to determine the doubling rate of the tumor cells.

Colony formation in soft agar is another measure of tumor growth. See Wu, Y. and D. Cai, Proc. Soc. Exp. Biol. Med., 201(3):284-288 (1992).

Tumorigenicity in an experimental animal can be measured by injecting an aliquot of cells, such as approximately 10⁶ cells, into an experimental animal subcutaneously and observing for tumor formation. See, Yeung, et al., J. Surg. Res. 53(20:203-210 (1992).

Phenotype refers to how the tumor looks, typically microscopically, but gross or macroscopic appearance can be observed. The phenotype changes depending on the growth rate of the tumor cells. For instance, the microscopic morphology of cells that are rapidly dividing and growing at a normal rate. Determination of tumor cell phenotype is well within the ability of one with ordinary skill in the art.

Thymidine incorporation can be a measure of tumor cell growth because thymidine is incorporated into rapidly growing cells at a higher rate than into static or less rapidly growing cells. See, Saito, et al., Eur. Arch. Otorhinolaryngol, 249(7):400-403 (1992): and Brooks, D. J. and Carewal, H. S., Int. J. Clin. lab. Res., 22(4): 196-200 (1992).

It will be readily apparent to those of ordinary skill in the art that a reduction or improvement in any one of the foregoing factors establishes that there has been a reduction in cell tumorigenicity.

### 5.7 Methods for Treatment of Cancer

Described below are methods and compositions for treating and/or preventing cancer using the ets2 gene or gene product as a therapeutic target. The outcome of a treatment is to at least produce in a treated subject a healthful benefit, which in the case of cancer, includes but is not limited to remission of the cancer, palliation of the symptoms of the cancer, control of metastatic spread of the cancer.

All such methods involve modulating ets2 gene activity and/or expression which in turn modulate the phenotype of the treated cell.

As discussed, above, successful treatment of cancer can be brought about by techniques which serve to decrease ets2 activity. Activity can be decreased by, for example, directly decreasing ets2 gene product activity and/or by decreasing the level of ets2 gene expression.

For example, compounds such as those identified through assays described, above, in Section 5.6, which decrease ets2 activity can be used in accordance with the invention to treat cancer. As discussed in Section 5.6, above, such molecules can include, but are not limited to peptides, including soluble peptides, and small organic or inorganic molecules, and can be referred to as ets2 antagonists. Dominant negative mutants of ets2 and ets2-repressor fusion proteins are also compounds that interfere with the interaction of ets2 with intracellular macromolecules may also be used. Techniques for the determination of effective doses and administration of such compounds are described, below, in Section 5.8.

Further, antisense and ribozyme molecules which inhibit ets2 gene expression can also be used in accordance with the invention to reduce the level of ets2 gene expression, thus effectively reducing the level of ets2 gene product present, thereby decreasing the level of ets2 activity. Still further, triple helix molecules can be utilized in reducing the level of ets2 gene activity. Such molecules can be designed to reduce or inhibit either wild type, or if appropriate, mutant target gene activity. Techniques for the production and use of such molecules are well known to those of skill in the art.

Any technique which serves to selectively administer nucleic acid molecules to a cell population of interest can be used, for example, by using a delivery complex. Such a delivery complex can comprise an appropriate nucleic acid molecule and a targeting means. Such targeting means can comprise, for example, sterols, lipids, viruses or target cell specific binding agents. Viral vectors that can be used with recombiant viruses include, but are not limited to adenovirus, adeno-associated virus, herpes simplex virus, vaccinia virus, and retrovirus vectors, in addition to other particles that introduce DNA into cells, such as liposomes.

### 5.7.1 Antisense Molecules

The use of antisense molecules as inhibitors of gene expression is a specific, genetically based therapeutic approach (for a review, see Stein, in Ch. 69, Section 5 “Cancer: Principle and Practice of Oncology”, 4th ed., ed. by DeVita et al., J.B. Lippincott, Philadelphia 1993). The present invention provides the therapeutic or prophylactic use of nucleic acids of at least six nucleotides that are antisense to a gene or cDNA encoding ets2 or a portion thereof. An “antisense” ets2 nucleic acid as used herein refers to a nucleic acid capable of hybridizing to a portion of a ets2 RNA (preferably mRNA) by virtue of some sequence complementarity. The invention further provides pharmaceutical compositions comprising an effective amount of the ets2 antisense nucleic acids of the invention in a pharmaceutically acceptable carrier, as described infra.

In another embodiment, the invention is directed to methods for inhibiting the expression of a ets2 nucleic acid sequence in a mammalian cell in vitro or in vivo comprising providing the cell with an effective amount of a composition comprising an ets2 antisense nucleic acid of the invention.

The antisense nucleic acid of the invention may be complementary to a coding and/or noncoding region of a ets2 mRNA. The antisense molecules will bind to the complementary ets2 gene mRNA transcripts and reduce or prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. The human ets2 promoter contains two CT repeats that represent potential triple helix regions (Mavrothalassitis et al., 1990, Oncogene 5:1337-1342). Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Nucleic acid molecules that are complementary to the 5′ end of the message, e.g., the 5untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335.

Nucleic acid molecules complementary to the 5′ untranslated region of the mRNA should include the complement of the AUG start codon. Antisense nucleic acid molecules complementary to mRNA coding regions are less efficient inhibitors of translation but could be used in accordance with the invention. Whether designed to to the 5′-, 3′- or coding region of target or pathway gene mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, at least 50 nucleotides, or at least 200 nucleotides. For example, in FIG. 6, nucleic acid molecules complementary to either the 5′- or 3′- non-translated, non-coding regions of the ets2 gene, could be used in an antisense approach to inhibit translation of endogenous ets2 gene mRNA.

Regardless of the choice of target sequence, it is preferred that in vitro studies are first performed to quantitate the ability of the antisense molecule to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense molecule can be DNA or RNA or chimeric mixtures or derivatives or modified versions thereof, single-stranded or double-stranded. The antisense molecule can be modified at the base moiety, sugar moiety, or phosphate backbone, for example, to improve stability of the molecule, hybridization, etc. The antisense molecule may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the antisense molecule may be conjugated to another molecule, e.g., a peptide, triggered cross-linking agent, transport agent, hybridization-triggered cleavage agent, etc.

The antisense molecule may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense molecule may also comprise at least one modified sugar moiety selected from the group including but not limited to arabinose, 2-fluoroarabinose, xylulose, and hexose.

In yet another embodiment, the antisense molecule comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense molecule is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Antisense molecules of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the ets2 coding region, such as the ones described in Section 6.1, could be used, those complementary to the transcribed untranslated region are also preferred.

The ets2 antisense nucleic acids can be used to treat or prevent formation of cancer involving a cell type that expresses, or preferably overexpresses, ets2. Cell types 10 which express or overexpress ets2 RNA can be identified by various methods known in the art. Such methods include but are not limited to hybridization with a ets2-specific nucleic acid (e.g., by Northern hybridization, dot blot hybridization, in situ hybridization), detection of ets2 gene product by immunoassays, etc. In a preferred aspect, primary tissue from a patient can be assayed for ets2 expression prior to treatment, e.g., by immunocytochemistry or in situ hybridization.

Pharmaceutical compositions of the invention comprising an effective amount of a ets2 antisense nucleic acid in a pharmaceutically acceptable carrier, can be administered to a patient having a disease or disorder which is of a type that expresses or overexpresses ets2 RNA or protein.

The amount of ets2 antisense nucleic acid which will be effective in the treatment of a particular disorder or condition will depend on the nature of the cancer or condition, and can be determined by standard clinical techniques. Where possible, it is desirable to determine the antisense cytotoxicity of the tumor type to be treated in vitro, and then in useful animal model systems prior to testing and use in humans.

The antisense molecules should be delivered to cells which express the ets2 gene in vivo. A number of methods have been developed for delivering antisense DNA or RNA to cells; e.g., antisense molecules can be injected directly into the tissue site, or modified antisense molecules, designed to target the desired cells (e.g., antisense molecule linked to peptides or antibodies that specifically bind receptors or antigens expressed on the target cell surface) can be administered systemically. Antisense molecules can be delivered to the desired cell population via a delivery complex. In a specific embodiment, pharmaceutical compositions comprising ets2 antisense nucleic acids are administered via biopolymers (e.g., poly-β-1->4-N-acetylglucosamine polysaccharide), liposomes, microparticles, or microcapsules. In various embodiments of the invention, it may be useful to use such compositions to achieve sustained release of the ets2 antisense nucleic acids. In a specific embodiment, it may be desirable to utilize liposomes targeted via antibodies to specific identifiable tumor antigens (Leonetti et al., 1990, Proc. Natl. Acad. Sci. U.S.A. 87:2448-2451; Renneisen et al., 1990, J. Biol. Chem. 265:16337-16342).

However, it is often difficult to achieve intracellular concentrations of the antisense sufficient to suppress translation of endogenous mRNAs. Therefore a preferred approach utilizes a recombinant DNA construct in which the antisense oligonucleotide or polynucleotide is placed under the control of a strong promoter, some of which are described in Section 5.3.1 supra. The use of such a construct to transfect target cells in the patient will result in the transcription of sufficient amounts of single stranded RNAs that will form complementary base pairs with the endogenous ets2 gene transcripts and thereby prevent translation of the ets2 gene mRNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced either directly into the tissue site, or via a delivery complex. Alternatively, viral vectors can be used which selectively infect the desired tissue. Any of the methods for gene therapy available in the art can be used. Exemplary methods are described below.

### 5.7.2 Ribozyme Molecules

Ribozymes are enzymatic RNA molecules capable of catalyzing the specific cleavage of RNA (For a review see, for example Rossi, J., 1994, Current Biology 4:469-471). The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins.

Ribozyme molecules designed to catalytically cleave ets2 gene mRNA transcripts can also be used to prevent translation of ets2 gene mRNA and expression of ets2 target genes. (See, e.g., PCT International Publication WO90/11364, published Oct. 4, 1990; Sarver et al., 1990, Science 247:1222-1225). While ribozymes that cleave mRNA at site specific recognition sequences can be used to destroy ets2 gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the ets2 gene mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively 30 described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in an ets2 gene.

As in the antisense approach, the ribozymes can be composed of modified oligonucleotides (e.g. for improved stability, targeting, etc.) and should be delivered to cells which express the ets2 gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous ets2 gene messages and inhibit translation. Because ribozymes unlike antisense molecules, are catalytic, a lower intracellular concentration is required for efficiency.

Anti-sense RNA and DNA, ribozynie, and triple helix molecules of the invention can be prepared by any method known in the art for the synthesis of DNA and RNA molecules. These include techniques for chemically synthesizing oligodeoxyri-bonucleotides and oligoribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. These nucleic acid constructs can be administered selectively to the desored cell population via a delivery complex.

Various well-known modifications to the DNA molecules can be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

### 5.7.3 Therapeutic Antibodies

Antibodies exhibiting capability to downregulate ets2 gene product activity can be utilized to treat cancer. Such antibodies can be generated using standard techniques described in Section 5.4, above, against full length wild type or mutant ets2 proteins, or against peptides corresponding to portions of the proteins such as, for example, the activation domains. The antibodies include but are not limited to polyclonal, monoclonal, Fab fragments, single chain antibodies, chimeric antibodies, and the like.

Because ets2 is an intracellular protein, it is preferred that internalizing antibodies be used. However, lipofectin or liposomes can be used to deliver the antibody or a fragment of the Fab region which binds to the ets2 gene product epitope into cells. Where fragments of the antibody are used, the smallest inhibitory fragment which binds to the ets2 activation domain(s) is preferred. For example, peptides having an amino acid sequence corresponding to the domain of the variable region of the antibody that binds to the ets2 activation domain(s) can be used. Such peptides can be synthesized chemically or produced via recombinant DNA technology using methods well known in the art (e.g., see Creighton, 1983, supra; and Sambrook et al., 1989, above). Alternatively, single chain antibodies, such as neutralizing antibodies, which bind to intracellular epitopes can also be administered. Such single chain antibodies can be administered, for example, by expressing nucleotide sequences encoding single-chain antibodies within the target cell population by utilizing, for example, techniques such as those described in Marasco et al. (1993, Proc. Natl. Acad. Sci. USA 90:7889-7893).

### 5.7.4 Gene Therapy

Gene therapy refers to treatment or prevention of cancer performed by the administration of a nucleic acid to a subject who has cancer or in whom prevention or inhibition of cancer is desirable. In this embodiment of the invention, the therapeutic nucleic acid produces intracellularly an antisense nucleic acid molecules that mediates a therapeutic effect by inhibiting ets2 expression. In another embodiment, nucleic acids comprising a sequence encoding a dominant negative mutant ets2 protein or non-functional fragment or derivative thereof, are administered to inhibit ets2 function by interfereing with the interactions of ets2 and with other molecules in the cell.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan,1993, Science 260:926-932; and Morgan and Anderson,1993, Ann. Rev. Biochem. 62:191-217; May,1993, TIBTECH 11(5):155-215). Methods commonly known in the art of recombinant DNA technology which can be used are described in Ausubel et al. (eds.),1993, Current Protocols in Molecular Biology, John Wiley & Sons, N.Y.; Kriegler, 1990, Gene Transfer and Expression, A Laboratory Manual, Stockton Press, N.Y.; and in Chapters 12 and 13, Dracopoli et al. (eds.), 1994, Current Protocols in Human Genetics, John Wiley & Sons, N.Y.

In one aspect, the therapeutic nucleic acid comprises a ets2 nucleic acid that is part of an expression vector that expresses a dominant non-functional ets2 protein or fragment or chimeric protein thereof in cancer cells. The function of ets2 is thought to be mediated by protein-protein interactions. Therefore, ets2 mutants that are defective in function but effective in binding to its interacting partner can be used as a dominant negative mutant to compete with the wild type ets2. Dominant non-functional ets2 can be engineered for expression in cancer cells that inappropriately overexpress ets2.

In a preferred aspect, the therapeutic nucleic acid comprises an antisense ets2 nucleic acid that is part of an expression vector that produces the antisense molecule in a suitable host. In particular, such a nucleic acid has a promoter operably linked to the antisense ets2 sequence, said promoter being inducible or constitutive, and, optionally, tissue-specific.

In another particular embodiment, a nucleic acid molecule is used in which the antisense ets2 sequences and any other desired sequences are flanked by regions that promote homologous recombination at a desired site in the genome, thus providing for intrachromosomal expression of the antisense ets2 nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector or a delivery complex, or indirect, in which case, cells are first transformed with the nucleic acid vitro, then transplanted into the patient. These two approaches are known, respectively, as in vivo or ex vivo gene therapy.

In a specific embodiment, the nucleic acid is directly administered in vivo, where it is expressed to produce the antisense nucleic acid molecule or encoded non-functional ets2 gene product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in biopolymers (e.g., poly-β-1->4-N-acetylglucosamine polysaccharide; see U.S. Pat. No. 5,635,493), encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); W092/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains the antisense ets2 nucleic acid is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The antisense ets2 nucleic acid to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdrl gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson,1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; and Mastrangeli et al.,1993, J. Clin. Invest. 91:225-234.

Adeno-associated virus (AAV) has also been proposed for use in gene therapy (Walsh et al.,1993, Proc. Soc. Exp. Biol. Med. 204:289-300.

The form and amount of therapeutic nucleic acid envisioned for use depends on the cancer, desired effect, patient state, etc., and can be determined by one skilled in the art.

A less preferred approach to gene therapy involves transferring an antisense ets2 gene or a dominant non-functional ets2 gene to cancer cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient, for purpose of replacing cells that are overexpressing ets2. In this embodiment, the nucleic acid is introduced into a cancer cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome mediated gene transfer, microcell-mediated gene transfer, spheroplast fusion, etc. Numerous techniques are known in the art for the introduction of foreign genes into cells (see e.g., Loeffler and Behr,1993, Meth. Enzymol. 217:599-618; Cohen et al., 1993, Meth. Enzymol. 217:618-644; Cline, 1985, Pharmac. Ther. 29:69-92). The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment,recombinant blood cells (e.g., hematopoietic stem or progenitor cells) are preferably administered intravenously.

Endogenous ets2 gene expression can also be reduced by inactivating or “knocking out” the gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional ets2 gene (or a completely unrelated DNA sequence) flanked by DNA homologous to the endogenous ets2 gene (either the coding regions or regulatory regions of the ets2 gene) can be used, with or without a selectable marker and/or a negative selectable marker, to transfect cells that express ets2 gene in vivo. Insertion of the DNA construct, via targeted homologous recombination, results in inactivation of the ets2 gene. Such approaches are particularly suited where modifications to ES (embryonic stem) cells can be used to generate animal offspring with an inactive ets2 gene (e.g., see Thomas & Capecchi 1987 and Thompson 1989, supra). Such techniques can also be utilized to generate animal models of cancer. It should be noted that this approach can be adapted for use in humans provided the recombinant DNA constructs are directly administered or targeted to the required site in vivo using appropriate viral vectors, e.g., herpes virus vectors.

Alternatively, endogenous ets2 gene expression can be reduced by targeting deoxyribonucleotide sequences complementary to the regulatory region of the ets2 gene (i.e., the ets2 gene promoter and/or enhancers) to form triple helical structures that prevent transcription of the ets2 gene in target cells in the body. (See generally, Helene, C. 1991, Anticancer Drug Des., 6(6):569-84; Helene, C., et al., 1992, Ann, N.Y. Acad. Sci., 660:27-36; and Maher, L. J., 1992, Bioassays 14(12):807-15).

### 5.8 Pharmaceutical Preparations and Methods of Administration

The compounds and nucleic acid sequences described herein can be administered to a patient at therapeutically effective doses to treat or prevent cancer. A therapeutically effective dose refers to that amount of a compound sufficient to result in a healthful benefit in the treated subject. Formulations and methods of administration that can be employed when the therapeutic composition comprises a nucleic acid are described in Section 5.8.2.

### 5.8.1 Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅vTD₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

### 5.8.2 Formulations and Use

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 5.9 Sensitizing Cancer Cells to Chemotherapy or Radiation Therapy

Many cancer cells are resistant to initial chemotherapeutic treatment or will eventually develop resistance to a chemotherapeutic agent. Some cancers respond poorly to treatment methods such as chemotherapy and radiation therapy (Boring et al., 1994, Cancer J. Clinic. 44: 7-26). As such, there is a need of sensitizing cancer cells so that these cells will be more receptive to treatment which improves treatment outcomes. It has been found that many instances of such resistance is related to the loss of DNA mismatch repair activity in cancer cells (Fink et al., 1998, Clinical Cancer Research, 4:1-6). When chemotherapeutic agents such as cisplatin, busulfan, temozolomide, and procarbazine are used to treat cancer, the varying degree of resistance of cancer cells to these drugs has been shown to produce a large difference in clinical responsiveness iii vivo as demonstrated in tumor model systems. Even though the mechanism for the establishment of drug resistance in cancer cells remains unknown, it is believed that many chemotherapeutic agents or radiotherapeutic agents act by forming adducts with DNA. Apparently, the cells respond to the presence of the DNA adduct by activating signal transduction pathways. For example, it has been shown that cisplatin activates c-jun amino-terminal kinase 1, c-Abl kinase, and mitogen-activated protein kinase (Persons et al., 1999, Clinical Cancer Res. 5:1007-1014). At present, it is unclear how signal transduction plays a role in drug resistance or sensitizing cancer cells.

Without being bound any theory, the inventors believe that ets2 gene expression and/or the activity of the ets2 protein is involved in one or more of the signal transduction pathways that are activated by the presence of lesions and abnormal structures in chromosomal DNA, such as DNA adducts. The methods and compositions as described in Section 5.7 supra can also be used to sensitize cancer cells to chemotherapeutic and radiotherapeutic treatment by interfering with such signal transduction pathways. Accordingly, the present invention provides methods for sensitizing cancer cells to chemotherapy or radiation therapy by down-regulating ets2 gene expression or ets2 activity.

Cancers, including, but not limited to, neoplasms, tumors, metastases, or any disease or disorder characterized by uncontrolled cell growth, that have been shown to be refractory to a chemotherapy or radiation therapy can be sensitized by administration of a therapeutic composition of the invention that modulates ets2 expression and/or function.

That a cancer is refractory to chemotherapy or radiation therapy means that at least some significant portion of the cancer cells are not killed or their cell division not arrested, by the particular chemotherapeutic agent or combination of chemotherapeutic agents or the level of radiation employed in a therapeutic protocol. The determination of whether the cancer cells are refractory to the chemotherapy or the radiation therapy can be made either in vivo or in vitro by any method known in the art.

In general, chemotherapy is carried out in cycles and only a certain percentage of cancer cells are killed during each round of chemotherapy or radiation therapy. However, if, after a round of chemotherapy or radiation therapy, the number of cancer cells has not been significantly reduced, or has increased, e.g., the size of a tumor remains the same or increased, then the cancer is refractory to that chemotherapy. And if subsequent rounds of chemotherapy or radiation therapy do not significantly reduce tumor load in the patient, then the cancer is refractory or resistant to that chemotherapy or radiation therapy.

Cancer cells can also be tested in vitro by culturing cancer cells removed from a patient, e.g., from a resected tumor. The cells can be contacted with various dosage of the chemotherapeutic agent or combination of the chemotherapeutic agents or the level of radiation used in the therapeutic protocol. If after the contact, there is no significant reduction in cancer cell number or results in an increase in cancer cell number (i.e., continued cell growth), then the cancer cells are refractory to such chemotherapy or radiation therapy.

In one embodiment of the invention, cancer cells that are refractory to radiation therapy are sensitized by administration of a composition of the invention. In another embodiment, the invention provides a method for sensitizing cancer cells with a composition of the invention, in which said cancer is refractory to treatment with a chemotherapeutic agent that kills or arrests the cancer cells in the S and/or M phases of the cell cycle. In a preferred embodiment, the methods and compositions of the present invention are used to sensitize cancer cells to treatment with any compound that induces the formation of adducts in DNA.

In various embodiments, cancer cells can be sensitized to the following chemotherapeutic agents, which can be divided generally into several categories according to their chemical properties and modes of action: the methylating agents; the alkylating agents; the platinum-containing drugs; the antimetabolites,; and the topoisomerase II inhibitors. Also useful are agents such as tamoxifen which act as an anti-estrogen (Jones et al., 1997, Cancer Res. 57:2657). Platinum-containing drugs like cisplatin and carboplatin can be used as a chemotherapeutic drug. When present in the eurkaryotic cells, they bind to their primary target and form adducts in the DNA (Fink et al., 1998, Clin. Cancer Res., 4:1-6). The structures of the aquated formed of cisplatin and carboplatin are the same, as are the types of adducts. Methylating agents, such as MNU, MNNG, procarbazine, temozolomide and dacarbazine form a variety of adducts in DNA, among which are O⁶-methylguanine, N⁷-methylguanine and N³-methyladenine DNA adducts. Alkylating agent, busulfan, also forms adduct with DNA. Antimetabolites such as 6-thioguanine and mercaptopurine are converted into 2′-deoxy-6-thioguanosine triphosphate and subsequently incorporated into DNA (Elion, 1989, Science 244: 41-47). After incorporation into DNA, 6-thioguanine can be chemically methylated by s-adenosylmethionine to form S⁶-methylthioguanine DNA adduct. Topoisomerase II inhibitors, such as etoposide and doxorubicin, are used in chemotherapy. These inhibitors bound to topoisomerase II which in turn, form a complex with DNA.

In particular embodiments, the methods and compositions of the present invention are used for the treatment or prevention of cancer together with one or a combination of chemotherapeutic agents including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel.

With respect to radiation therapy, any radiation therapy protocol can be used depending upon the type of cancer to be treated. For example, but not by way of limitation, X-ray radiation can be administered; in particular, high-energy megavoltage (radiation of greater than 1 MeV energy) can be used for deep tumors, and electron beam and orthovoltage x-ray radiation can be used for skin cancers. Gamma ray emitting radioisotopes, such as radioactive isotopes of radium, cobalt and other elements may also be administered to expose tissues. Radiation is known to cross-linked DNA.

The methods of sensitizing cancer cells comprise modulating the ets2 gene activity and/or expression concurrently with chemotherapy or radiation therapy. In another embodiment, chemotherapy or radiation therapy is administered, preferably at least an hour, five hours, 12 hours, a day, a week, a month, more preferably several months (e.g., up to three months), subsequent to using the methods and compositions containing the ets2 gene or gene product. In a less preferred embodiment, chemotherapy or radiation therapy is administered before using the methods and compositions containing the ets2 gene or gene product. The chemotherapy or radiation therapy administered prior to, concurrently with, or subsequent to the treatment using the methods and compositions containing ets2 gene or gene product, can be administered by any method known in the art. The chemotherapeutic and radiotherapeutic agents are preferably administered in a series of sessions.

As used herein, that the cancer cells are sensitized means that by comparison to cells not treated by the methods of the invention, more cancer cells or a significant number of cancer cells are killed or cancer cell division is arrested, or the size of a tumor or metastasis is reduced, or the size of a colony is reduced, or the cancer cells exhibit a less aggressive phenotype, by treatment with a particular dose of chemotherapeutic and/or radiotherapeutic agent(s), within the same amount or a shorter period of time. When, the cancer cells are sensitized, the same number of cells can be killed with a lower dose of a chemotherapeutic agent or a combination therefor, or a lower level of radiation employed in a therapeutic protocol. The sensitized cancer cells may become less prone to become resistant to a chemotherapeutic agent resulting in fewer drug resistant clones. The drug resistance of the cancer cells may be reversed by treatment with the methods of the invention.

In another embodiment, the methods of sensitizing cancer cells with a therapeutic composition of the invention can be used in combination with a significantly lower level or shorter session of chemotherapy and/or radiation therapy where the chemotherapy or the radiation therapy has proven or may prove too toxic, i.e., results in unacceptable or unbearable side effects, for the subject being treated.

To determine whether the cancer cells are sensitized to chemotherapy or radiation therapy, any method known in the art, either in vivo or in vitro, for assaying the effectiveness of treatment on cancer cells can be used. The sensitivity of cancer cells can be determined by various methods that are known in the art which include, but are not limited to, measuring apoptosis and the levels of p53 and Bcl-2 expression (Wu et al., 1996, Clin. Cancer Res., 2(4):623-33), and measuring DNA synthesis as a percentage of inhibition of DNA synthesis by a anti-cancer agent (Kawabata et al., 1998, Anticancer Res, 18(3A):1633-40). The sensitivity of cancer cells can also be determined by many in vivo chemosensitivity tests including, but not limited to, succinic dehydrogenase inhibition test (Ishimura, 1996, Hokkaido Igaku Zasshi, 71(6):689-98), collagen gel-droplet embedded culture drug sensitivity test (CD-DST)(et al., 1997, Int. J. Oncol., 11:449), conventional SDI test (Ogihara et al., 1996, Nippon Hinyokika Gakkai Zasshi, 87(4):740-7), adenosine triphosphate (ATP) assay, diphenyltetrazolium bromide (MTT) test, (Shi et al., 1996, Chung Hua Fu Chan Ko Tsa Chih, 31(2):79-82), clonogenic assays and micronucleus assay using a cytokinesis-block in which maximal percentage of binucleate cells or multinucleate cells are determined at various chemotherapeutic agent concentrations (Jeremi'c et al., 1996, Srp Arh Celok Lek, 124(7-8):169-74).

### 6. Example: ETS2 and Prostate Cancer

Prostate cancer cells which overexpress ets2 was chosen for detailed characterization. This example demonstrates the expression of ets2 mRNA in human prostate cancer cell lines, the correlation of ets2 expression with the transformed phenotype of the prostate cancer cell lines, and the reversal of the transformed phenotype by blockage of ets function. The example also showed that a reduction of the transformed phenotype of the prostate cancer cells in vitro is associated with reduced tumorigenicity in an animal model.

### 6.1. Materials and Methods

Cell Lines, Tissue Culture and DNA Transfection

The human prostatic carcinoma cell lines LNCaP, DU145 and PC3 were obtained from American Type Culture Collection (Rockville, Md.) and were propagated at 37° C., with 5% C02 in RPMI 1640 supplemented with 10% fetal bovine serum (FBS). The LNCaP cell line originated from prostatic tumor cells metastasized to a supraclavicular lymph node. It has been found to possess chromosomal deletions at 7q22, 10q24 and 16q22 and contains a wild type p53 gene. It has a well-differentiated phenotype, is androgen sensitive and PSA and prostatic acid phosphatase positive. Based upon these properties, LNCaP has been used as a model for well-differentiated prostate cancer. LNCaP is weakly tumorigenic and not metastatic after subcutaneous injection into nude mice. The DU145 line was derived from a brain metastasis. It has an aneuploid number of chromosomes (between 61 and 65), and has a deletion of the RB gene and a mutation in p53. It is androgen insensitive, PSA negative and is tumorigenic. The PC3 line was derived from a bone metastasis (grade IV adenocarcinoma), and is aneuploid, with a mean number of chromosomes ranging from 55 to 58. The PC3 cell line is mutated in p53 and is tumorigenic. The properties of DU145 and PC3 suggest that they represent poorly differentiated, aggressive prostate cancer. Cell lines were tested for mycoplasma contamination and were not infected.

Transfection of LNCaP was performed in 35 mm wells using 2 μg of DNA and Superfact Reagent (Qiagen, Calif.). Selection for stable LNCaP transfectants was in 450 μg/ml G418. DU145 and PC3 cells were transfected using LipofectAmine (10 μl/35 mm well, Life Technologies Inc., Bethesda, Md.). Stable DU145 and PC3 transfectants were expanded in the presence of 300 μg/ml G418.

Soft agar growth assays were performed in six-well plates (35-mm wells). Cells (20,000) were plated in 0.4% agar (Sigma) in RPMI 1640 supplemented with 10% FBS. An underlayer of 0.8% agar was used to prevent attachment of cells. Triplicate assays initiated with 20,000 cells were fed weekly by agar overlay and scored after 3 weeks. The soft agar assays were performed twice, independently.

Tumorigenicity in SCID Mice

Tumorigenicity of the prostatic cell lines was examined by s.c. inoculation of 5×10⁵ cells into four week old SCID mice.

Expression Vectors

ETS2 cDNA (Sstll to HindIII fragment from pK3A (Watson et al., 1988, Proc Nati Acad Sci USA, 85:7862-6) was subcloned in antisense and sense orientation into the eukaroytic expression vectors, pSGneoKS and pSGneoSK [modifications of pSG5 (Stratagene, La Jolla, Calif,) containing a neomycin/G418-resistance cassette and the multiple cloning site from pBluescript II KS or SK vectors, respectively]. The pK3A vector, primers and Pfu polymerase were used to PCR amplify a portion of the human ETS2 cDNA encoding the DNA binding domain (C-terminal amino acids, 334-469). The 5′ primer (GAGAACTAGTACCACCATGGATTACATCCAAGAGA GGA) contains the recognition sequence for Spel and a Kozak consensus start sequence in frame with aspartic acid residue 334 of the ETS2 cDNA to allow for efficient protein translation. The 3′ primer (GAGACTCGAGGCGACCTCAGTCCTCCGTGTC) contains the recognition sequence for Xhol and the translation termination codon from the ETS2 m gene. The resultant PCR product was digested with Spel and Xhol and cloned in the sense orientation between the Spel and XhoI sites of the pSGneoSK to generate the pDN-ETS2 vector. Orientation and sequence of all constructs were verified by sequence analysis (ABI 373).

RNA Extraction and Analysis

RNA from cultured cells was purified using RNAStat (Tel-Test, Inc., Friendswood, Tex.). Total RNA was fractionated on 1.2% agarose gels containing 0.66 M formaldehyde (2.2 M in the sample) by the method of Lehrach et al (Lehrach et al., 1977, Biochemistry, 16:4743-4751). Gels were transferred to Duralon filters (Stratagene) in 0.1 M sodium phosphate (pH 6.8), UV cross-linked, and hybridized in Quik-Hyb (Stratagene) using ³²p labeled human ETS2 probe (Watson et al., 1988, Proc Natl Acad Sci USA, 85:7862-6).

### 6.2. Results

### 6.2.1. Expression of ETS2 mRNA in human prostate cell lines

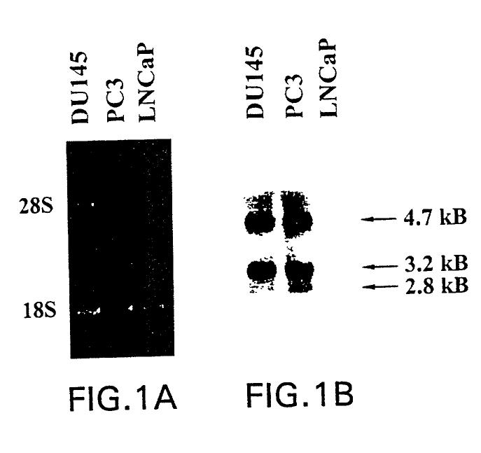

To initiate our analysis of the role of ETS2 in prostatic cancer, we examined the expression of ETS2 in three commonly used human cell lines derived from prostate cancer. We prepared RNA from three cell lines (LNCaP, DU145 and PC3). Northern analysis demonstrates that the ETS2 mRNA products (4.7, 3.2 and 2.8 kb) are expressed at significant levels in the DU145 and PC3 cell lines, while they are not present at detectable levels in LNCaP (FIG. 1). This observation is consistent with the hypothesis that ETS2 expression is associated with cell lines representing a more aggressive phenotype.

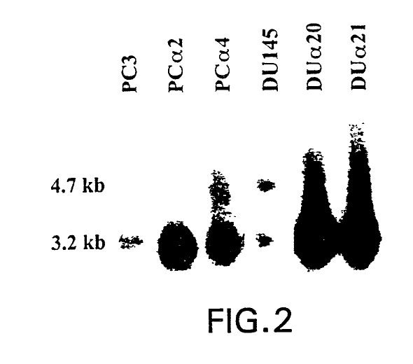

To assess whether ETS2 expression is related to the transformed state of prostate cancer cells, we performed an mRNA “knockdown” experiment by transfection of DU145 and PC3 cells prostatic cancer cells with a plasmid that constitutively expresses an antisense ETS2 RNA. Following selection in G418, individual clones were screened by northern blot hybridization for the presence of the exogenous ETS2 mRNA. DU145 and PC3 antisense ETS2 clones that demonstrated a decrease in the expression of the endogenous ETS2 4.7 kb MRNA species (FIG. 2A; Note: The antisense RNA comigrates with the 3.2 kb mRNA) were chosen for further analysis.

### 6.2.2. ETS2 Expression Correlates with the Transformed Phenotype of DU145 and PC3 Prostate Cancer Cells

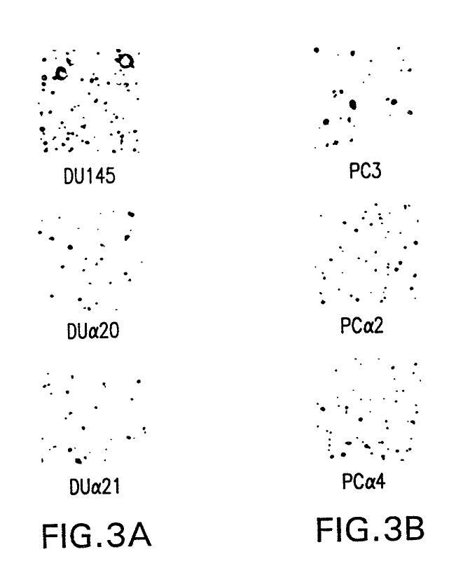

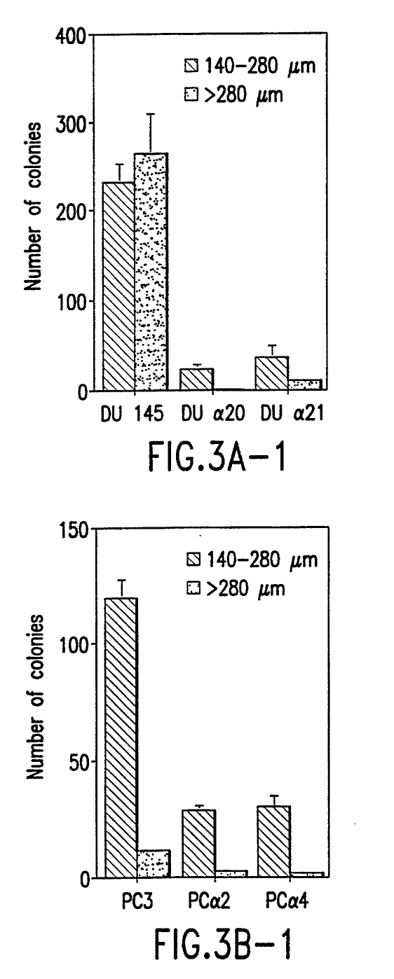

Two clones expressing the antisense RNA and no longer expressing the endogenous ETS2 message (FIG. 2), along with each parental cell line, were tested for anchorage independent growth, a cellular phenotype that is correlated with in vivo tumorigenicity. The ability of DU145 and PC3 to form large colonies in soft agar was significantly decreased in the antisense transfectants (FIG. 3). After three weeks in soft agar, the transfectants have a reduced ability to form large colonies. The quantitative data on the colony numbers shows that that the reduction of large (>280 μm) colonies is significantly greater than for small colonies (140-280 μm). These changes in colony formation in soft agar do not appear to be related to growth rate, since the transfected cells and parental cells have similar growth rates when grown on plastic.

### 6.2.3. Reversal of Transformed Phenotype of Prostate Cancer Cells by Blockage of ETS Function

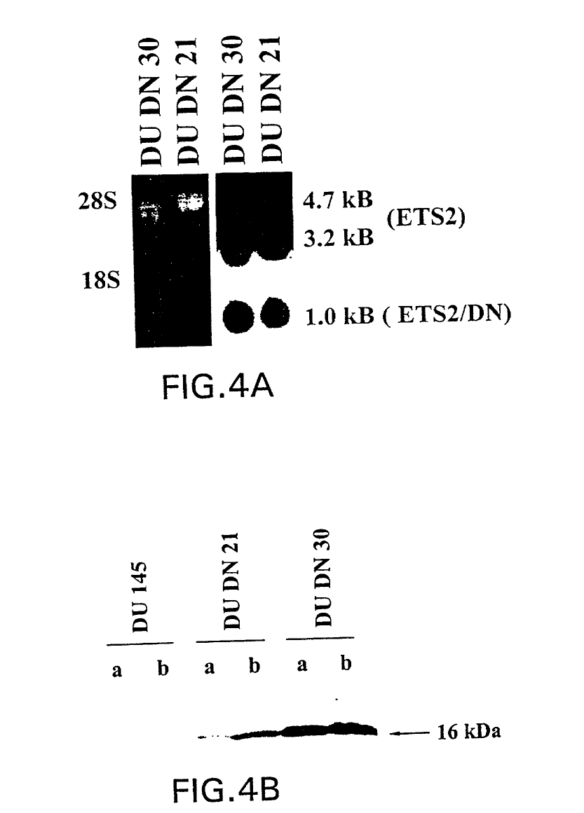

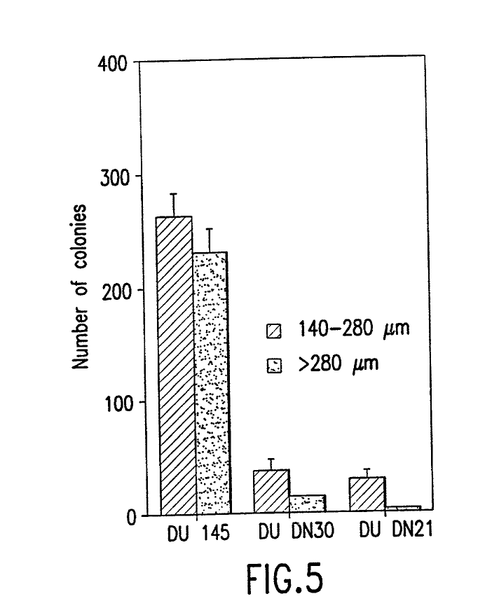

Expression of the DNA binding domain of Ets transcription factors in the absence of their transactivation domain has been shown to suppress the transcription of Ets target genes (Langer et al., 1992, Mol Cell Biol, 12:5355-62 and Wasylyk et al., 1994, Oncogene, 9:3665-73). A DNA segment encoding the C-terminal 133 amino acids of the human ETS2 protein, including the DNA binding domain and sequences required for nuclear localization, was cloned into a modified pSG5 eukaryotic expression vector under the control of the SV40 promoter. The plasmid, pDN-ETS2, was transfected into DUI45 and PC3 cells and independent stable cell lines were obtained. Stable transfectants were analyzed for the presence of the exogenous 1.0 kb ETS2 mRNA by northern blot hybridization (FIG. 4a). DU145 and PC3 transfectants expressing the DN-ETS2 transcript were expanded and compared with the parental cell lines for the presence of the truncated ets2 protein. Radioimmunoprecipitation analyses were performed using two different ETS antibodies recognizing the DNA binding carboxy terminal domain. Both antibodies (FIG. 4b, pan ets (a) and C20(b)] precipitated a protein of approximately 16 kDa from two transfectants, but not from the parental cells. This size is consistent with the predicted size of 15,960 Da. These two independent clones were compared with the parental cell line for anchorage-independent growth. Compared with the DU145 parental cell lines, the DN-ETS2 transfectants do not grow well in soft agar (FIG. 5). Thus, inhibiting ETS2 function reverses the transformed phenotype of prostate cancer cell lines.

### 6.2.4. Antisense ETS2 Inhibits Tumorigenicity of PC3 in SCID Mice

The ability of cells to grow in an anchorage-independent manner has been shown to correlate with in vivo tumorigenicity. To determine whether ETS2 expression affects the tumorigenicity of prostate cell lines, we compared the phenotype of mice injected with PC3 parental cells with that of mice injected with an antisense ETS2 transfectant of PC3 (PC(×2). Subcutaneous inoculation of the parental PC3 (5×10⁵ cells ) into SCID mice results in the formation of large tumors (1×1×1 cm and 1.4×1×1.2 cm) within 6 weeks. Tumors grew toward the peritoneal cavity, without grossly violating the peritoneal cavity. The tumors were not excessively vascularized. The animals also became extremely wasted and lost approximately 50% of their body mass. In contrast, animals injected with an antisense ETS2 transfectant [PCα2] developed only tiny palpable tumors and otherwise were unremarkable.

### 5 6.2.5. Increased Expression of ETS2 in Two Drug-Resistant Cell Lines

To initiate our analysis of the role of ETS2 and ETS1 in drug-resistance, we examined the expression of ets2 and etsl transcripts in a melanoma cell line 289, and two related drug-resistant cell lines, 289T (tamoxifen-resistant) and 289D (cisplatin-resistant) (McClay et al., 1996, Cancer Res. 57:3993-3997). RNA was prepared from the three cell lines 289, 289T and 289D. Total RNA (5 μg/lane) was electrophoresed on 1.2% agarose containing formaldehyde, transferred to a nylon membrane and hybridized with ³²P-labeled ETS2 and ETS1 probes. Northern analysis demonstrates that the ETS2 mRNA products are expressed at significant levels in the drug-resistant 289T and 289D cell lines, relative to the parental 289 cell line. This observation is consistent with the hypothesis that ETS2 expression is associated with cell lines that are less sensitive to chemotherapeutic drugs. The ETS1 mRNA products are expressed at significant levels in all three cell lines.

### 6.2.6. ETS2 Confers Resistance to Cisplatin

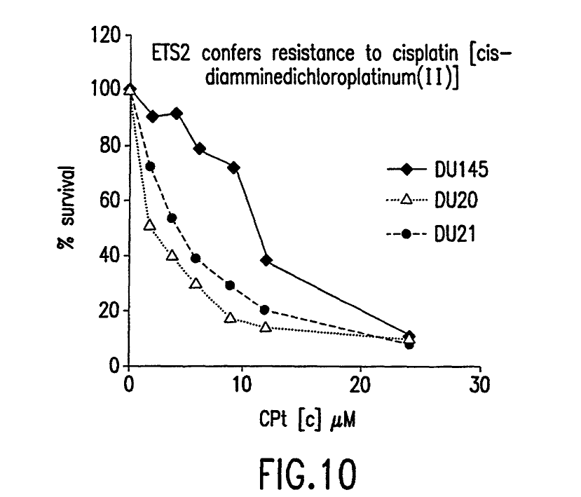

To determine whether cancer cells can be sensitized to chemotherapy by modulating the ets2 gene activity and/or expression, we performed a cell sensitivity assay. DU145 cells, which is derived from human prostate cancer, were transfected with a plasmid that constitutively expresses antisense ets2 RNA molecules. Following selection in G418, individual clones were screened by Northern blot hybridization for the presence of the antisense ets RNA and reduction of endogenous ets2 mesenger RNA. Two independent stable cell lines were obtained. These transfectants, DU20 and DU21, expressing the antisense ets2 RNA molecules, together with the DU145 cancer cells were subjected to different concentrations of cisplatin [cis-diamminedichloroplatinum (11)] and their percentage survival were measured and compared (FIG. 10). Compared with the DU145 parental cell line, the antisense ets2 transfectants, DU20 and DU21, have a lower percentage survival at all concentrations between zero to approximately 25 μM of cisplatin.

### 6.3. Discussion

The results described above show that the ets2 gene is up-regulated in prostate cancer tissues and cell lines, and that antisense RNA-induced inhibition of expression of ets2 in prostate cancer cell lines dramatically reduces the ability of these cells to form anchorage independent colonies in soft agar. These observations support the idea that ets2 expression in prostate epithelia contributes to the transformed state in prostate cancer.

The goal of this study was to assess the contribution of one member of the ETS gene family, ETS2, in prostate cancer. Transfection provides a model system in which we can manipulate ETS2 expression in prostate cancer cells to determine its contribution to the transformed phenotype. Two approaches to block ETS2 function in DU145 and PC3 human prostate cell lines that express ETS2 have been developed. We have generated clonal cell lines that express an antisense construct and consequently no longer express the endogenous ETS2 transcripts. In addition, we have inhibited ETS2 function as a transcription factor by creating prostate cell clones expressing a transdominant negative mutant that is likely to compete with endogenous ETS2 protein for Ets binding sites. Cell lines that no longer have functional ETS2 have a much reduced ability to grow in an anchorage independent compared to the parental cell lines. We have also demonstrated that de novo expression of ETS2 into the weakly tumorigenic-LNCaP human prostate cell line increases its ability to grow in soft agar. The results presented support the model that ETS2 function(s) are necessary for the maintenance of the transformed phenotype of prostate cancer cells and that dysregulation of the ETS2 gene control contributes to aggressive prostate cancer. Furthermore, ETS2 may play a role in controlling genes that are misexpressed in aggressive cancer and our cell lines provide a system will allow identification of Ets target genes that are associated with cancer progression. Interestingly, DU145 and PC3 (ETS2 expressing) prostate cancer cell lines are invasive, while LNCaP (not expressing ETS2) are not, as measured by migration through Matrigel coated membranes (Wasilenko et al., 1996, International Journal of Cancer, 68:259-64). Elevated expression of ETS2 in invasive cells is consistent with Ets function in the regulation of stromelysin (Gutman, A. and Wasylyk, B., 1990, Ernbo J. 9:2241-6) and collagenase (Wasylyk et al., 1991, Embo J, 10:1127-34), enzymes that degrade extracellular matrix. Plasminogen activator urokinase (u-PA), an Ets target (Nerlov et al., 1991, Embo J, Oncogene, 6:15883-92) is necessary for in vitro invasiveness and metastasis of PC3 cells (Crowley et al.,1993, Proceedings of the National Academy of the Sciences, USA, 90:5021-5025). Other Ets target genes have been found to be upregulated in prostate cancer and function to promote cell proliferation, motility and angiogenesis, properties that play critical roles in carcinogenic progression. For example, c-met, the receptor for hepatocyte growth factor/scatter factor, is regulated by Ets (Gambarotta et al., 1996, Oncogene, 13:1911-7) and the presence of met protein has been correlated with higher grade adenocarcinomas (Pisters et al., 1995, Journal of Urology, 154:293-8). Mitogenic signalling through the ErbB/neu receptor is mediated through Ets (Langer et al., 1992, Mol Cell Biol, 12:5355-62 and Galang et al., 1996, Journal of biological Chemistry, 271:7992-8) and elevated neu expression is associated with metastatic conversion of prostate cancer (Zhau et al., 1992, Molecular Carcinogenesis, 5:320-7 and Zhau et al., 1996, Prostate, 28:73-83). In addition to regulating genes over-expressed in prostate cancer cells, Ets has recently been shown to be a regulate maspin, a tumor suppressing protease inhibitor that is expressed in normal prostate epithelial cells and down-regulated in prostate cancer cell lines (Zhang et al., 1997, Proceedings of the National Academy of the Sciences, USA, 94:5673-8). Lower expression of maspin in LNCaP cells was due in part to the loss of Ets-mediated transcriptional activation (Zhang et al., 1997, Proceedings of the National Academy of the Sciences, USA, 94:5673-8). Collectively, these observations demonstrate the importance of Ets target genes in prostate cancer.

Due to the similarity of Ets binding sites, it is quite likely that the DN-ETS2 mutant can inhibit the transcriptional activity of other ETS family members. Thus, in addition to inhibiting ETS2 transcriptional targets, it is expected that the DN-ETS2 mutant would also block the transcriptional activity of other ETS family members that are expressed in prostatic cancer cell lines (e.g., ETS1, unpublished observations). Thus, interference of Ets function may provide a novel therapeutic approach for cancers that overexpress Ets family genes.

The feasibility of using gene therapy to treat cancer has also been tested. The strategy is based on delivering antisense ets2 nucleic acid molecules to cancer cells in a patient which causes downregulation of endogenous ets2 gene expression in vivo, and results in tumor regression in the patient. The above described results suggested that an antisense ets2 nucleic acid molecule can be delivered to prostate cancer cells by use of an adenovirus-based vector system, and that it could cause a change in the phenotype of the infected cancer cells. Moreover, the result obtained in the SCID mouse model correlates with observations made in the in vitro soft agar growth assay, and indicates that prostate cancer cells in which ets2 expression is inhibited by antisense RNA are less tumorigenic in vivo.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

In the claims:

1. A modified ets2 protein which consists essentially of the amino acid sequence from position 361 to position 446 as set forth in FIG. 7.

2. A modified ets2 protein that comprises a repressor element, binds an ets2 target site and lacks transcriptional activation activity.

3. The modified ets2 protein of claim 2 wherein the repressor element is selected from the group consisting of the KRAB box, and the SNAG domain.

4. A nucleic acid molecule comprising a nucleotide sequence encoding the modified ets2 protein of claim 1, 2, or 3, operably associated with at least one regulatory region that controls expression of the modified ets2 protein in a cell.

5. A recombinant cell containing a nucleotide sequence encoding the modified ets2 protein of claim 1, 2, or 3, operably associated with at least one regulatory region that controls expression of the modified ets2 protein in the cell.

6. The recombinant cell of claim 5 wherein the cell is a cancer cell.

7. The recombinant cell of claim 6 wherein the cell is a human cell.

8. A method for preparing a modified ets2 protein comprising:
(a) culturing the recombinant cells of claim 5; and
(b) recovering from the cell culture the modified ets2 protein.

9. The method of claim 8 further comprises purifying the modified ets2 protein.

10. An antisense ets2 nucleic acid.

11. A nucleic acid comprising a nucleotidc sequence encoding an antisense ets2 nucleic acid, operably associated with at least one regulatory region that controls expression of the antisense ets2 nucleic acid in a cell.

12. An ets2-specific ribozyme, comprising an ets2-specific binding site and an autocatalytically cleaving portion.

13. A nucleic acid comprising a nucleotide sequence encoding an ets2-specific ribozyme, which ribozyme comprising at least a portion of an ets2-specific binding site and an autocatalytic cleaving ribozyme.

14. A delivery complex comprising the nucleic acid molecule of claim 12 and a targeting means.

15. The delivery complex of claim 14 wherein the targeting means is selected from the group consisting of a sterol, a lipid, a virus, or a target cell specific binding agent.

16. A method for diagnosing a cancer in a human subject comprising detecting or measuring a ets2 gene product in a subject, in which an elevated level of the ets2 gene product relative to a reference level indicates the existence of cancer.

17. The method of claim 16 in which the cancer is prostate cancer.

18. The method of claim 16 in which the cancer is lung cancer, pancreas cancer. prostate cancer, liver cancer, testicular cancer, ovarian cancer, cervical cancer, or breast cancer.

19. The method of claim 16 wherein the cancer involves metastases.

20. A method of treating or preventing cancer comprising administering an effective amount of a compound that decreases the expression of ets2.

21. The method of claim 20 wherein the compound comprises an antisense ets2 nucleic acid or ribozyme molecule, that decreases the expression of ets2 gene.

22. A method of treating or preventing cancer comprising administering to a subject an effective amount of a compound that antagonizes the activity of ets2.

23. The method of claim 22 wherein the compound is a modified ets2 protein that comprises a repressor element, that binds an ets2 target site and lacks transcriptional activation activity.

24. A method of treating or preventing cancer comprising administering an effective amount of a DNA molecule expressing an antisense ets2, a ribozyme molecule, or a triplex molecule.

25. The method of claims 16, 20, 22, and 24 further comprising the step of using chemotherapy and/or radiation therapy.

26. A method of reducing tumorigenicity or metastatic potential of cancer cells comprising administering to the individual an effective amount of a compound that decreases the expression of ets2 gene.

27. A method of reducing tumorigenicity or metastatic potential of cancer comprising administering an effective amount of a compound that antagonizes the activity of ets2 protein.

28. A method of sensitizing cancer cells to a cancer treatment or prevention method in an individual having cancer or in whom treatment or prevention of cancer is desired comprising administering to the individual an effective amount of modified ets2 protein.

29. A method of sensitizing cancer cells to a cancer treatment or prevention method in an individual having cancer or in whom treatment or prevention of cancer is desired comprising administering to the individual a therapeutic effective amount of antisense ets2 nucleic acid.

30. The method of claims 16, 22, 24, 26, 27, 28, and 29 in which the cancer is selected from the group consisting of pancreatic cancer, breast cancer, ovarian cancer, prostate cancer, liver cancer, cervical cancer, testicular tumor, lung carcinoma, and melanoma.

31. The method of claim 26, 27, 28, or 29 in which the cancer treatment or prevention method is selected from the group consisting of chemotherapy and radiation therapy.

32. The method of claim 28 or 29 wherein said cancer treatment or prevention method employs an alkylating agent, methylating agent, platinum-containing agent, antimetabolite or topoisomerase II inhibitor.

33. The method of claim 28 or 29 wherein said cancer treatment or prevention method employs tamoxifen, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, or docetaxel.

34. The method of claim 23 in which the repressor element in the modified ETS2 protein is selected from the group consisting of the KRAB box, and the SNAG domain.

35. The method of claim 23 in which the modified ets2 protein consists essentially of the amino acid sequence from position 361 to position 446 as set forth in FIG. 7.

36. The method of claim 20, 21, 22, 23, 24, 28, or 29, in which the cancer has metastasized.

37. The method of claim 26, 27, 28, or 29 in which the cancer is refractory to treatment with an agent selected from the group consisting of alkylating agent, methylating agent, platinum-containing agent, antimetabolite and topoisomerase II inhibitor.

38. The method of claim 28 or 29, in which the cancer is melanoma and the cancer treatment or prevention method employs cisplatin.

39. The method of claim 28 or 29, in which the cancer is melanoma and the cancer treatment or prevention method employs tamoxifen.
